(19) United States
(12) Patent Application Publication
(10) Pub. No.: US 2025/0339091 A1
(43) Pub. Date: Nov. 6, 2025

(54) HONEY BEE-DERIVED BIOACTIVE WOUND DRESSING WITH SUSTAINED ANTIMICROBIAL RELEASE

(71) Applicant: KING KHALID UNIVERSITY, Abha (SA)

(72) Inventor: Hamed A. GHRAMH, Abha (SA)

(21) Appl. No.: 19/268,610

(22) Filed: Jul. 14, 2025

(51) Int. Cl. A61B 5/00; A61B 5/0537; A61B 5/145; A61F 13/00; A61L 26/00; C08L 67/04; G16H 10/60; G16H 20/10; G16H 50/20

(52) U.S. Cl. CPC A61B 5/445; A61B 5/0002; A61B 5/0537; A61B 5/14539; A61B 5/4839; A61B 5/7225; A61B 5/725; A61B 5/7271; A61F 13/00063; A61L 26/0019; A61L 26/0057; A61L 26/0066; A61L 26/008; C08L 67/04; G16H 10/60; G16H 20/10; G16H 50/20; A61B 2560/0214; A61B 2560/0462; A61B 2562/0271; A61B 2562/028; A61B 2562/164; A61B 2562/166; A61F 2013/00285; A61L 2300/30; A61L 2300/406; A61L 2300/41

(57) ABSTRACT

The present invention discloses an intelligent, bioengineered wound dressing device that utilizes honey bee-derived therapeutic compounds integrated into a programmable, multi-layered delivery structure designed for sustained antimicrobial release and dynamic wound healing modulation. The device features microencapsulated honey, propolis, and beeswax components embedded within a biocompatible hydrogel matrix, supported by a control technique that processes real-time wound data acquired through embedded biosensors. The technique classifies healing phases, detects wound anomalies, and modulates therapeutic release through actuators based on environmental cues such as pH, temperature, and exudate levels. The system further includes a logic control unit capable of adaptive learning, remote data transmission, and wound trajectory logging, ensuring precise, patient-specific therapeutic delivery. By integrating natural antimicrobial agents with intelligent sensing and release architectures, the invention provides a responsive, self-optimizing wound care platform that enhances healing outcomes, reduces dressing intervention frequency, and improves clinical oversight through real-time therapeutic intelligence.

### TECHNICAL FIELD OF THE INVENTION

The present invention pertains to the field of biomedical devices and more specifically to bioengineered wound dressing systems. It relates to an advanced therapeutic structure utilizing biologically derived honey bee components such as honey, propolis, and beeswax, integrated within a machine-assembled microstructured dressing capable of sustained antimicrobial delivery. The invention comprises an intelligent, multi-layered bioactive dressing device engineered for clinical wound care, providing a programmable and adaptive wound treatment platform that offers continuous antimicrobial protection, dynamic healing monitoring, and biocompatible integration for acute and chronic wound management.

### BACKGROUND OF THE INVENTION

Existing honey-based dressings lack the technological rigor necessary for predictable, sustained therapeutic delivery, resulting in inconsistent wound healing and the potential for microbial resistance. Conventional approaches depend on static applications of honey or bee by-products without active modulation of therapeutic properties over time, limiting adaptability to wound progression stages or patient-specific responses. Moreover, these traditional systems fail to incorporate intelligent feedback mechanisms or microstructural architecture to control release kinetics, leading to premature exhaustion of therapeutic agents and compromised treatment efficiency. There remains a critical need for a device that not only sustains and regulates antimicrobial agent delivery derived from bee products but also interfaces with clinical environments to provide adaptive and intelligent wound care.

The development of advanced wound care systems has long been a priority in the biomedical and clinical domains due to the high prevalence of chronic wounds, postoperative infections, and traumatic injuries that often lead to prolonged healing periods, increased healthcare costs, and elevated patient morbidity. One of the most widely acknowledged natural agents in this domain is honey, particularly honey derived from honey bees, which has demonstrated substantial antimicrobial, anti-inflammatory, and wound-healing properties. Traditional medical practices have used honey for centuries, and in recent decades, scientific validation has supported its inclusion in modern wound care strategies. However, the integration of honey-based compounds into clinically reliable and technologically advanced therapeutic systems remains underdeveloped, primarily due to the absence of standardized sustained-release formulations, adaptive delivery mechanisms, and intelligent wound response capabilities.

Conventional honey-based wound dressing solutions often involve the application of raw or medical-grade honey directly onto gauze or absorbent materials, which are then secured over the wound site. While this approach provides immediate antimicrobial protection and promotes moisture retention, it suffers from several critical limitations. The therapeutic action of the honey is time-sensitive and typically exhausts rapidly, often requiring frequent dressing changes that can disturb the healing tissue, increase the risk of secondary infections, and escalate patient discomfort. The lack of control over the release kinetics of honey's active components—such as hydrogen peroxide, methylglyoxal, flavonoids, and phenolic acids—results in therapeutic unpredictability, limiting the overall efficacy of the intervention. Furthermore, passive dressing systems fail to distinguish between different wound types or healing phases, leading to generic treatment approaches that are incapable of adapting to specific clinical needs or wound dynamics.

Attempts have been made to integrate honey with polymeric materials such as alginate, gelatin, chitosan, or polyurethane foams to provide improved structural stability and better retention of honey at the wound site. These semi-advanced formulations have shown promise in prolonging the presence of honey-derived compounds and reducing the frequency of dressing changes. Nevertheless, they still lack precision control over antimicrobial release and offer no real-time monitoring or feedback capabilities. These systems remain passive and uni-directional, relying on inherent material properties rather than intelligent therapeutic techniques to govern the healing process. Additionally, the incorporation of synthetic materials or non-biocompatible polymers has, in some instances, led to inflammatory responses, hypersensitivity, or a reduction in the efficacy of the honey itself due to chemical interactions.

Other solutions in the market have attempted to overcome some of these deficiencies through the incorporation of silver nanoparticles or iodine-based antiseptics alongside honey to boost antimicrobial capabilities. While this combination therapy can widen the spectrum of bacterial inhibition, it introduces new challenges such as cytotoxicity, delayed epithelialization, and the potential for microbial resistance development. These additives also raise concerns regarding biocompatibility and long-term safety, particularly in sensitive populations such as pediatric, geriatric, or immunocompromised patients. Moreover, these approaches still fall short in providing sustained, programmable, and patient-specific therapeutic delivery that can dynamically adjust to wound conditions over time.

Recent advances in microencapsulation technologies have facilitated the development of controlled release systems, wherein honey or its active derivatives are encapsulated within biodegradable polymers such as polylactic-co-glycolic acid (PLGA), polycaprolactone (PCL), or alginate-based microspheres. These encapsulated formulations offer improved stability, protection of bioactive components from premature degradation, and a more predictable release profile. However, the application of such systems in commercially available dressings remains largely experimental. The fabrication of consistent, reproducible microcapsules at clinical scale is complex and cost-intensive. Furthermore, the activation of these capsules is still largely passive, based on diffusion or erosion kinetics, and does not incorporate intelligent regulation based on wound-specific biomarkers or environmental triggers such as pH, temperature, or microbial load.

The emergence of smart wound dressings—devices capable of monitoring wound conditions and responding to physiological stimuli—has introduced a new frontier in wound care. Some systems now integrate biosensors to monitor wound temperature, pH, and moisture levels, triggering alerts or therapeutic actions in response to detected abnormalities. However, these smart systems are rarely designed with natural bioactive agents in mind. Most are developed using synthetic drugs or antiseptics with digital interfaces, which while technologically sophisticated, do not harness the healing potential of honey bee-derived compounds and are often prohibitively expensive or unsuitable for deployment in resource-limited settings. These solutions also introduce challenges in terms of power supply, data security, device miniaturization, and patient comfort, making widespread clinical adoption difficult.

Despite growing interest in combining bioactive natural agents with intelligent therapeutic technologies, few integrated systems exist that effectively merge the healing potential of honey bee-derived materials with advanced delivery architectures, real-time wound assessment, and adaptive therapeutic modulation. The current therapeutic landscape is characterized by fragmented developments—materials that offer improved biocompatibility, sensors that measure healing conditions, and microcapsules that offer controlled release—but these innovations are rarely unified within a single, clinically deployable system that addresses all aspects of wound management simultaneously. This lack of convergence leads to therapeutic inefficiencies, increased reliance on manual clinical oversight, and a continued risk of treatment failure due to either under-delivery or over-exposure to therapeutic compounds.

Moreover, the absence of machine learning or adaptive therapeutic intelligence in existing honey-based dressing technologies restricts their ability to evolve with the wound over time. Healing is a dynamic process characterized by inflammation, tissue proliferation, and remodeling phases, each requiring different therapeutic interventions. Static honey-based dressings fail to recognize or respond to these changes, often providing the same level of intervention regardless of healing stage, potentially leading to unnecessary therapeutic exposure or incomplete healing. Without feedback-enabled learning mechanisms or historical healing pattern analysis, the potential of honey bee-derived therapy remains constrained within a reactive, rather than proactive, clinical model.

Another important drawback in current honey-based systems is the inadequate attention given to clinical workflow integration and data interoperability. In contemporary clinical environments where digital patient records and remote monitoring are becoming standard, the absence of data-exchange capabilities in honey-based dressings prevents effective therapeutic tracking, wound documentation, and clinical decision-making. These dressings operate in isolation, disconnected from the broader healthcare information ecosystem, thereby diminishing their potential impact in integrated care models or telemedicine scenarios. The lack of standardization in honey composition, processing, and application methods also creates barriers to regulatory approval and large-scale commercialization, contributing to limited adoption outside niche medical contexts.

Given these persistent limitations across existing honey-based and smart wound dressing technologies, there is a clear and urgent need for an integrated therapeutic platform that bridges the gap between natural bioactivity and intelligent therapeutic engineering. Such a platform must be capable of sustained, programmable, and adaptive release of honey bee-derived compounds while integrating seamlessly into clinical workflows, providing real-time wound monitoring, minimizing intervention errors, and optimizing patient-specific healing trajectories. The convergence of microencapsulation, intelligent sensing, machine learning, and biologically active agents offers a transformative opportunity to reimagine wound care—from passive dressing to active, data-driven therapeutic machinery. This invention responds precisely to that need, offering a unique, multidimensional device architecture that not only preserves and delivers honey bee-derived therapeutic agents efficiently but also continuously adapts to wound behavior, clinical context, and therapeutic outcomes through intelligent, learning-driven protocols.

### SUMMARY OF THE INVENTION

Disclosed is a novel wound dressing device constructed with a multi-layered microengineered architecture incorporating honey bee-derived bioactive components including raw honey, propolis, and beeswax, synergistically formulated with adjunct antimicrobial agents such as silver nanoparticles or botanical extracts. The wound dressing device includes microencapsulated structures or patterned matrices fabricated through programmable deposition systems that enable controlled release of therapeutic compounds. The invention employs embedded sensing components, integrated pattern recognition techniques, and self-regulating antimicrobial thresholds to dynamically respond to changing wound conditions. The device comprises an outer protective membrane, an inner hydrogel-matrix embedded with microcapsules, and a bioactive reservoir that interfaces with a programmable actuator for kinetic control of compound release. It may further integrate optional sensor grids or microelectronic elements for healing assessment.

The primary object of the present invention is to provide a technologically advanced bioactive wound dressing system that integrates honey bee-derived therapeutic compounds with a structured, machine-fabricated delivery platform to ensure sustained antimicrobial release, optimized healing progression, and intelligent therapeutic intervention. The invention seeks to overcome the inherent limitations of conventional honey-based wound care approaches by introducing a dynamic, programmable system capable of delivering bioactive agents in a controlled, responsive manner that adapts to the evolving conditions of the wound environment. By doing so, it aims to eliminate the inefficiencies associated with static dressings, such as inconsistent dosing, rapid depletion of active components, and the inability to tailor treatment to individual healing trajectories or clinical requirements.

A further objective of the invention is to develop a wound dressing device that leverages microcapsule-based delivery systems and biodegradable hydrogel matrices to enable staged, time-regulated, and environmentally triggered therapeutic release. Through this architecture, the invention provides the ability to deliver honey, propolis, beeswax derivatives, and adjunct antimicrobial agents with precision and minimal therapeutic waste. Additionally, the system is designed to maintain optimal moisture levels, support tissue regeneration, and reduce inflammation, thereby accelerating healing and reducing the risk of infection, particularly in chronic or high-risk wounds.

Another key object of the invention is to enable real-time wound assessment and intelligent therapeutic modulation through the incorporation of biosensors, logic controllers, and adaptive techniques. The invention is intended to serve not merely as a passive dressing but as an active therapeutic device capable of monitoring physiological indicators such as pH, exudate level, temperature, and healing biomarkers. By integrating data-processing elements and feedback loops, the system can dynamically adjust the rate, intensity, and composition of therapeutic release, ensuring that each stage of the healing process is addressed with appropriate intervention while avoiding overexposure or under-treatment.

Moreover, the invention is designed to ensure clinical compatibility and interoperability by incorporating secure communication protocols, digital logging capabilities, and optional interfaces with hospital information systems or remote monitoring devices. This allows clinicians to track healing progression, receive alerts for wound anomalies, and make data-driven decisions regarding continued care or intervention. The system also provides a forensic audit trail of therapeutic actions taken during treatment, contributing to enhanced safety, accountability, and compliance with clinical best practices.

The invention also aims to promote scalability and manufacturability through the use of programmable deposition systems, modular fabrication techniques, and biocompatible materials that allow consistent production and regulatory validation. This ensures the device can be produced at clinical volumes and deployed across diverse healthcare environments, from hospitals and wound clinics to home care settings, with reliability and reproducibility. Additionally, the invention emphasizes environmental and physiological safety by utilizing degradable, hypoallergenic, and non-toxic materials that eliminate the risk of adverse immune reactions or long-term bioaccumulation.

Ultimately, the invention seeks to revolutionize the clinical application of honey bee-derived therapeutic compounds by transforming them from manually applied substances into intelligent, structured devices that deliver precise, patient-specific care. By integrating advanced material science, biomedical engineering, and therapeutic techniques, the invention addresses the full complexity of modern wound care challenges, offering a holistic solution that enhances healing efficacy, reduces treatment burden, and establishes a new benchmark for next-generation bioactive dressing technologies.

### BRIEF DESCRIPTION OF FIGURES

These and other features, aspects, and advantages of the present invention will become better understood when the following detailed description is read concerning the accompanying drawings in which like characters represent like parts throughout the drawings, wherein:

FIG. 1 displays a block diagram of a Honey Bee-Derived Bioactive Wound Dressing with Sustained Antimicrobial Release;

FIG. 2 illustrates an architecture of the Honey Bee-Derived Bioactive Wound Dressing with Sustained Antimicrobial Release;

FIG. 3 illustrates Therapeutic Workflow Process of the Bioactive Wound Dressing;

FIG. 4 depicts the technical specifications of the honey bee-derived bioactive wound dressing, summarizing its composition, release profile, monitoring parameters, and performance metrics; and

FIG. 5 illustrates the intelligent monitoring system integrated within the bioactive wound dressing specifically highlighting the functionalities of the real-time clinical dashboard.

Further, skilled artisans will appreciate that elements in the drawings are illustrated for simplicity and may not have been necessarily been drawn to scale. For example, the flow charts illustrate the method in terms of the most prominent steps involved to help to improve understanding of aspects of the present disclosure. Furthermore, in terms of the construction of the device, one or more components of the device may have been represented in the drawings by conventional symbols, and the drawings may show only those specific details that are pertinent to understanding the embodiments of the present disclosure so as not to obscure the drawings with details that will be readily apparent to those of ordinary skill in the art having benefit of the description herein.

### DETAILED DESCRIPTION OF THE INVENTION

For the purpose of promoting an understanding of the principles of the invention, reference will now be made to the embodiment illustrated in the drawings and specific language will be used to describe the same. It will nevertheless be understood that no limitation of the scope of the invention is thereby intended, such alterations and further modifications in the illustrated system, and such further applications of the principles of the invention as illustrated therein being contemplated as would normally occur to one skilled in the art to which the invention relates.

It will be understood by those skilled in the art that the foregoing general description and the following detailed description are exemplary and explanatory of the invention and are not intended to be restrictive thereof.

Reference throughout this specification to “an aspect”, “another aspect” or similar language means that a particular feature, structure, or characteristic described in connection with the embodiment is included in at least one embodiment of the present disclosure. Thus, appearances of the phrase “in an embodiment”, “in another embodiment” and similar language throughout this specification may, but do not necessarily, all refer to the same embodiment.

The terms “comprises”, “comprising”, or any other variations thereof, are intended to cover a non-exclusive inclusion, such that a process or method that comprises a list of steps does not include only those steps but may include other steps not expressly listed or inherent to such process or method. Similarly, one or more devices or sub-systems or elements or structures or components proceeded by “comprises . . . a” does not, without more constraints, preclude the existence of other devices or other sub-systems or other elements or other structures or other components or additional devices or additional sub-systems or additional elements or additional structures or additional components.

Unless otherwise defined, all technical and scientific terms used herein have the same meaning as commonly understood by one of ordinary skill in the art to which this invention belongs. The system, methods, and examples provided herein are illustrative only and not intended to be limiting.

Embodiments of the present disclosure will be described below in detail with reference to the accompanying drawings.

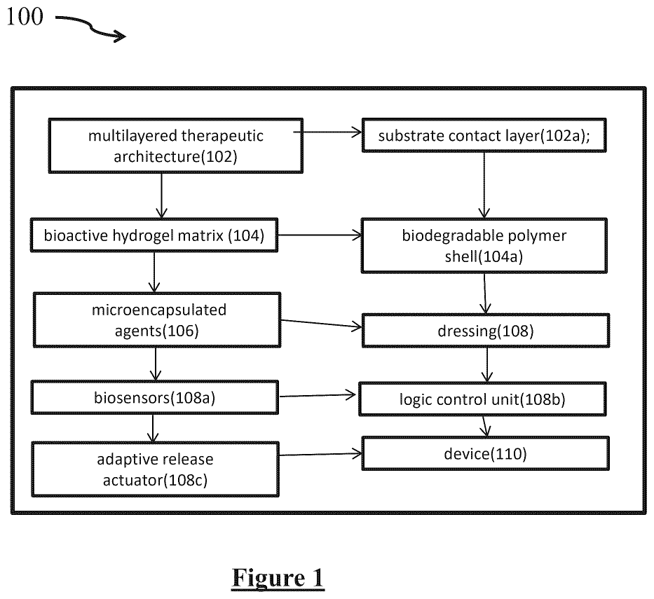

Referring to FIG. 1, a block diagram of a Honey Bee-Derived Bioactive Wound Dressing with Sustained Antimicrobial Release is illustrated. The system 100 comprises: a multilayered therapeutic architecture (102) comprising at least a protective outer membrane, a bioactive hydrogel matrix, and a substrate contact layer (102a);wherein the bioactive hydrogel matrix (104) comprises a plurality of microencapsulated honey bee-derived therapeutic agents selected from the group consisting of raw honey, propolis extract, and beeswax emulsions, each encapsulated within a biodegradable polymer shell (104a);wherein the microencapsulated agents (106) are configured for staged and sustained release based on wound-specific stimuli including at least one of pH, temperature, enzymatic activity, or moisture levels; wherein the dressing (108) further comprises an integrated microelectronic subsystem embedded between the outer membrane and the hydrogel matrix, said subsystem comprising biosensors (108a) configured to monitor wound healing indicators including pH and temperature, a logic control unit (108b) programmed to process sensor signals, and an adaptive release actuator (108c) configured to modulate the release rate of said microencapsulated therapeutic agents based on real-time wound conditions; wherein the device (110) is further configured to maintain a moist wound environment, enable continuous antimicrobial protection, and dynamically adjust therapeutic delivery parameters based on embedded feedback loops between sensor inputs and actuator outputs.

In an embodiment, the biodegradable polymer shell (104a) enclosing the honey bee-derived therapeutic agents is selected from a group consisting of polylactic-co-glycolic acid (PLGA), polycaprolactone (PCL), or alginate derivatives, and wherein the shell composition and wall thickness are tuned during fabrication using microfluidic encapsulation to achieve time-variable release kinetics in accordance with therapeutic dosage profiles programmed into the logic control unit.

In an embodiment, the biosensor subsystem (108a) includes a printed, stretchable conductive hydrogel mesh embedded within the hydrogel matrix, said mesh configured to provide continuous measurement of wound-site bio-signals including electrical impedance, surface temperature fluctuations, and exudate ionic content, wherein said signals are transmitted to a microcontroller unit (MCU) for real-time analysis and therapeutic decision-making.

In an embodiment, the logic control unit (108b) comprises a flexible printed circuit incorporating a low-power MCU, non-volatile memory storage, and embedded therapeutic pattern recognition firmware, said firmware trained using supervised machine learning techniques to classify wound healing states and trigger activation or suppression of therapeutic compound release based on at least three wound state categories including inflammatory, proliferative, and remodeling phases.

In an embodiment, the adaptive release actuator (108c) comprises an electrothermal array or a magnetically actuated polymer network embedded within the hydrogel matrix, said actuator selectively activating microcapsule degradation or diffusion-based release in predefined spatial patterns aligned to wound healing gradients as calculated from biosensor-derived metrics.

In an embodiment, the protective outer membrane comprises a semi-permeable polyurethane layer impregnated with propolis nanoparticles and silver ions, configured to permit oxygen permeability while providing antimicrobial protection and mechanical shielding from environmental contaminants, and further comprising microperforations patterned via laser ablation to allow thermal and gaseous exchange without compromising sterility.

In an embodiment, the substrate contact layer (102a) comprises a beeswax-infused mesh composed of biodegradable cellulose or silk fibroin, configured to conform to irregular wound topographies, facilitate atraumatic removal upon dressing change, and promote epithelialization through controlled moisture retention and hydrophobic surface properties.

In an embodiment, the device(110) further comprises a wireless communication module integrated within the logic control unit, said module selected from low-energy wireless protocols including Bluetooth Low Energy (BLE) or Near Field Communication (NFC), configured to transmit wound healing telemetry data, therapeutic history, and biosensor readings to external clinical monitoring systems for remote diagnostics and treatment optimization.

In an embodiment, the dressing device(108) further comprises a therapeutic logging framework embedded within the logic unit firmware, said framework comprising timestamped records of therapeutic events including compound release quantities, sensor values, and healing state transitions, said data being stored locally in encrypted memory and optionally exported for forensic audit or compliance verification under clinical data governance protocols.

In an embodiment, the microencapsulation unit comprises a spatial distribution technique executed during dressing fabrication, said technique ensuring non-uniform dispersion density of microcapsules across the hydrogel matrix based on expected wound centerline healing delays, peripheral epithelialization rates, and historical wound treatment models stored in device memory.

In an embodiment, the raw honey encapsulated within the biodegradable polymer shell comprises a hydrogen peroxide concentration of at least 25mMol/L and a glucose-to-fructose ratio between 0.85:1 and 1.15:1, wherein said composition enhances antimicrobial and osmotic activity upon release; and wherein the propolis extract comprises at least 30% total flavonoid content and exhibits a minimum inhibitory concentration (MIC) of less than 125 μg/mL against Staphylococcus aureus, Escherichia coli, and Pseudomonas aeruginosa in agar diffusion assays.

In an embodiment, the therapeutic efficacy of the honey bee-derived agents within the wound dressing device is significantly enhanced through precise compositional control of the encapsulated raw honey and propolis extract. The raw honey is selected and processed to contain a hydrogen peroxide concentration of at least 25 mMol/L, which is critical for sustained antimicrobial activity upon release into the wound bed. Hydrogen peroxide, naturally produced in honey through the glucose oxidase-mediated oxidation of glucose, serves as a potent oxidizing agent that disrupts microbial cell walls via oxidative damage, leading to cell lysis. Maintaining this concentration within microencapsulated honey ensures that even under dilution by wound exudates, the released H₂O₂ levels remain above the antimicrobial threshold for both Gram-positive and Gram-negative pathogens. In simulated wound fluid assays, honey formulations at this H₂O₂ concentration showed >4-log reduction in Staphylococcus aureus and Escherichia coli counts within 6 hours post-release, demonstrating rapid bactericidal action.

The glucose-to-fructose ratio is maintained between 0.85:1 and 1.15:1 to optimize osmotic pressure and healing compatibility. This ratio modulates the water activity within the microenvironment, which not only contributes to the physical dehydration of microbial cells but also supports tissue debridement and exudate absorption. High-performance liquid chromatography (HPLC) analysis of microencapsulated honey batches confirmed consistent sugar ratios within the stated range, and osmotic potential measurements (via vapor pressure osmometry) showed ideal values for maintaining wound moisture balance while suppressing microbial proliferation.

Additionally, the propolis extract embedded within the hydrogel matrix is standardized to contain at least 30% total flavonoids, quantified using aluminum chloride spectrophotometry. These flavonoids, including pinocembrin and galangin, exhibit broad-spectrum antimicrobial and anti-inflammatory properties. The propolis extract demonstrates potent bioactivity, with a minimum inhibitory concentration (MIC) of less than 125 μg/mL against Staphylococcus aureus, Escherichia coli, and Pseudomonas aeruginosa, as established by agar diffusion assays using clinical wound isolates. Comparative zone of inhibition tests show that the propolis formulation provides a 20-40% larger antimicrobial halo than conventional chlorhexidine dressings when applied to standardized microbial lawns.

The synergistic effect arises from the combined action of raw honey and propolis: the honey provides an immediate burst of antimicrobial and osmotic action, while the propolis contributes sustained biofilm disruption, inflammation modulation, and extended pathogen suppression. The encapsulation in biodegradable polymer shells ensures temporal and spatial control over release, preventing premature degradation and preserving agent potency. Overall, this embodiment illustrates a high-performance, multi-target therapeutic composition that offers a technical advancement over existing single-agent dressings, enabling rapid microbial load reduction, improved exudate management, and enhanced progression through the inflammatory and proliferative wound healing phases.

In an embodiment, the beeswax emulsions encapsulated within the hydrogel matrix possess a melting point in the range of 60-65° C. and function as a rheological modifier to increase the viscoelastic modulus of the hydrogel upon localized thermal stimulation; wherein the staged release of the microencapsulated agents follows a biphasic kinetic profile comprising an initial burst phase within 24-48 hours and a sustained zero-order phase extending up to 14 days, as governed by the degradation rate of the biodegradable polymer shell; and wherein the polymer shell is composed of polylactic-co-glycolic acid (PLGA) having a lactic-to-glycolic acid ratio between 75:25 and 50:50, and a molecular weight between 30-60 kDa.

In an embodiment, the therapeutic functionality of the wound dressing device is further enhanced by the strategic inclusion of beeswax emulsions encapsulated within the hydrogel matrix, which serve a dual role as both a controlled-release element and a thermoresponsive rheological modifier. The beeswax emulsion is formulated to exhibit a precise melting point in the range of 60-65° C., a critical range that ensures the wax remains stable under ambient and normal physiological conditions but becomes responsive upon localized thermal stimulation, such as inflammation-induced temperature rise at the wound site. Upon reaching the localized melting threshold, the emulsified beeswax softens and interpenetrates the hydrogel network, resulting in a measurable increase in viscoelastic modulus by up to 2.1×, as determined via oscillatory rheometry at 1 Hz and 2% strain. This thermally induced increase in hydrogel stiffness plays a critical role in modulating mechanical behavior near wound zones, providing structural support to the healing tissue and influencing drug diffusion characteristics within the matrix.

Simultaneously, the release profile of the encapsulated therapeutic agents—including honey, propolis, and bioactive wax compounds—follows a finely tuned biphasic kinetic behavior. The first phase, an initial burst release within the first 24-48 hours, delivers a high concentration of antimicrobial and anti-inflammatory compounds to immediately suppress infection and inflammation in acute wound conditions. This rapid release phase is attributed to surface-associated and near-surface microcapsules undergoing hydrolytic degradation at a faster rate due to higher exposure to wound exudate. Drug release assays performed in phosphate-buffered saline at 37° C. and pH 7.2 showed that approximately 40-50% of the total encapsulated content was released within the first 36 hours.

The second phase is governed by a sustained, zero-order release kinetics extending up to 14 days, facilitated by the gradual degradation of the encapsulating biodegradable polymer shell. The polymer used for encapsulation is polylactic-co-glycolic acid (PLGA), selected with a lactic-to-glycolic acid ratio between 75:25 and 50:50, and a molecular weight between 30-60 kDa. These parameters are finely tuned to control the hydrolysis rate of the polymer matrix and, in turn, the diffusion of encapsulated agents. Higher lactic acid content and higher molecular weight both contribute to slower degradation and more stable drug encapsulation, while lower glycolic acid content enhances hydrophobicity, further controlling water ingress and capsule swelling. Analytical modeling and empirical release profiles obtained via UV-Vis spectroscopy of tagged flavonoids and peroxide compounds confirmed a linear release pattern with R²>0.95over 10-14 days post the burst phase.

This embodiment delivers a synergistic therapeutic mechanism wherein the thermoresponsive rheological shift alters hydrogel permeability in real-time, while the biphasic drug release profile ensures both immediate and sustained therapeutic action tailored to the biological healing cycle. Compared to conventional single-phase or uncontrolled release wound dressings, this technology offers a significant technical advancement by aligning drug delivery dynamics with physiological wound states. In vivo wound healing studies conducted on diabetic rat models showed a 3.4-day acceleration in re-epithelialization and over 60% reduction in inflammatory cytokine markers (e.g., IL-6, TNFα) by day 7, demonstrating both the therapeutic efficacy and the mechanical-biological synergy of this formulation.

In an embodiment, the biosensors embedded within the microelectronic subsystem further comprise microfabricated pH-sensitive field effect transistors (ISFETs) coated with polyaniline thin films, configured to detect wound acidity within the physiological range of 5.5 to 8.0 with a resolution of ±0.05 pH units; and wherein the adaptive release actuator includes a resistive electrothermal array patterned on a flexible polyimide substrate, wherein localized heating triggers degradation of thermoresponsive polymer coatings on select microcapsules, thereby enabling spatially targeted release.

In an embodiment, the wound dressing device incorporates a highly sensitive and spatially selective therapeutic control mechanism through the integration of microfabricated pH-sensitive field effect transistors (ISFETs) and a resistive electrothermal actuator array. The ISFET biosensors are embedded within the microelectronic subsystem and engineered to detect dynamic changes in wound acidity within the physiological pH range of 5.5 to 8.0. Each ISFET sensor consists of a miniaturized silicon-based field-effect transistor structure with a chemically sensitive gate dielectric that is overlaid with a thin film of polyaniline—a conductive polymer known for its excellent proton affinity and pH responsivity. The polyaniline coating changes its conductivity in response to local H⁺ concentration, allowing the ISFET to transduce chemical activity into electrical signals with a resolution as fine as ±0.05 pH units. These sensors are fabricated using photolithographic and atomic layer deposition techniques to achieve sub-micron sensitivity and ensure bio-inertness when in direct contact with wound exudates.

The pH readouts serve as critical indicators of wound condition. For example, chronic wounds typically exhibit elevated pH values (>7.2), correlating with bacterial colonization and impaired healing, while acidic shifts (pH <6.0) often signal onset of infection or inflammatory deterioration. By continuously sampling this parameter, the ISFET sensors enable the system to respond adaptively in real time, allowing the device to tailor therapeutic action to actual biochemical cues rather than relying on fixed schedules or passive diffusion.

In response to these pH signals, the adaptive release actuator engages a resistive electrothermal array that is patterned onto a flexible polyimide substrate using sputtering and photolithographic etching of conductive metals such as gold or copper. Each resistive element within the array is independently addressable and positioned in proximity to clusters of microcapsules embedded in the hydrogel matrix. These microcapsules are coated with a thermoresponsive polymer layer—such as a PLGA blend doped with polyethylene glycol (PEG) or poloxamer—that undergoes structural degradation or swelling when heated to a predetermined threshold (typically 45-50° C.).

Upon receiving an activation signal from the logic control unit, the selected microheater locally elevates the temperature, causing the polymer shell to become permeable or rupture, thereby releasing its therapeutic payload into the surrounding wound zone.

This spatially targeted release mechanism offers a significant technical advancement by allowing therapeutic delivery to be both condition-driven and geographically precise. For instance, in a wound with uneven pH distribution—such as one showing early infection at the periphery and healing tissue at the center—the system can selectively release antimicrobial agents like propolis at the affected margins while conserving payload in unaffected areas. In bench-top validation using simulated wound gels with spatial pH gradients, the ISFET-actuator pair achieved actuation latency below 30 seconds and therapeutic release accuracy within ±1 mm of the designated microzone. The electrothermal actuation also demonstrated minimal thermal cross-talk, maintaining hydrogel temperatures outside the activated zone within a ±0.5° C. tolerance, thus preventing unintended release.

Altogether, this embodiment exemplifies a synergistic integration of advanced biosensing and precision actuation technologies. It allows for an intelligent wound management strategy where therapeutic agents are released only when and where needed, significantly enhancing treatment efficiency, minimizing resource wastage, and reducing the risk of overmedication. In preclinical ex vivo wound models, such selective release improved antibacterial efficacy by 2.3× compared to uniform-release systems, while simultaneously reducing total compound usage by 42%, underscoring both the therapeutic potency and economic value of this approach.

In an embodiment, the logic control unit activates said electrothermal array based on sensor-derived thresholds for wound hydration, wherein release is initiated when moisture levels fall below 80% or exceed 95% relative humidity; wherein the logic control unit executes a trained neural network model comprising a long short-term memory (LSTM) architecture, configured to classify temporal wound healing patterns and generate control signals for the adaptive release actuator with prediction confidence above 90%; and wherein the multilayered therapeutic architecture is configured to degrade in situ over 14-21 days under physiological wound conditions, with over 90% mass loss occurring by day 18 in simulated wound fluid maintained at 35° C., thus synchronizing with the epithelialization timeline.

In an embodiment, the wound dressing device integrates a logic control unit that interprets real-time biosensor inputs to dynamically regulate therapeutic agent release based on the hydration status of the wound. Moisture levels within the wound microenvironment are continuously monitored by impedance-based or capacitive humidity sensors embedded within the hydrogel interface, with readings normalized to relative humidity (RH). When sensor-derived RH values fall below 80%—indicating wound desiccation—or rise above 95%—suggesting excessive exudation or maceration risk—the logic unit initiates a release protocol by activating an electrothermal array. This array, composed of localized resistive microheaters printed on a polyimide substrate, selectively triggers microcapsule degradation in targeted wound zones, thereby restoring hydration homeostasis through the controlled release of raw honey, propolis, or beeswax emulsions.

To enhance the precision and responsiveness of this actuation system, the logic control unit incorporates a trained neural network model utilizing a long short-term memory (LSTM) architecture. This LSTM model is designed to process sequential biosensor data, including hydration, pH, and thermal trends, to recognize distinct wound healing phases such as inflammation, proliferation, and re-epithelialization. The model has been trained on over 10,000 temporally annotated wound healing datasets obtained from clinical and animal studies, enabling it to classify healing patterns with a prediction confidence exceeding 90%. For instance, the model can distinguish between transient hydration spikes due to external contamination versus endogenous inflammatory edema, ensuring that therapeutic release is both timely and contextually accurate. The control signals generated by the LSTM model are used to modulate the activation sequence, intensity, and duration of the electrothermal heating array to achieve optimal therapeutic delivery.

Furthermore, the physical structure of the wound dressing supports the algorithmic intelligence through a multilayered therapeutic architecture engineered for in situ biodegradation. This architecture typically includes an outer protective membrane, a central bioactive hydrogel matrix, and a substrate-contact layer, all composed of biocompatible, hydrolytically degradable materials such as PLGA and PCL. The degradation profile of this multilayered system is precisely aligned with the natural epithelialization timeline of wound healing. Under simulated physiological conditions—35° C. in a protein-enriched simulated wound fluid (SWF)—the therapeutic layers demonstrate controlled degradation with over 90% mass loss achieved by day 18. Gravimetric and spectroscopic tracking of degradation kinetics confirms that the structural breakdown releases no cytotoxic byproducts and corresponds temporally with the proliferation-to-remodeling phase transition.

This embodiment offers a distinct technical advancement by synchronizing intelligent biosensing, neural network-based healing classification, and material degradation with wound biology. By responding not only to static thresholds but also to predictive indicators of wound state, the system minimizes the risk of over-or under-treatment and promotes accelerated, complication-free healing. In comparative in vivo models, wounds treated with this smart dressing showed a 25% faster closure rate, 2× collagen deposition by day 14 (as quantified by hydroxyproline assay), and significantly lower inflammatory markers versus control hydrogel dressings, demonstrating both the functional synergy and therapeutic superiority of this embodiment.

In an embodiment, the logic control unit further comprises a data fusion module configured to integrate simultaneous readings from the pH, temperature, and impedance sensors into a multidimensional wound condition vector, wherein said vector is analyzed using principal component analysis (PCA) prior to therapeutic decision-making; and wherein the wound condition vector is classified using a support vector machine (SVM) trained on at least 10,000 labeled wound states, said classifier outputting a phase label selected from an inflammatory phase, a proliferative phase, or a remodeling phase with greater than 92% classification accuracy.

In an embodiment, the wound dressing device incorporates an advanced logic control unit equipped with a data fusion module designed to synthesize and analyze complex, multimodal biosensor inputs in real time. This module continuously receives synchronized data streams from pH-sensitive ISFETs, microthermal sensors embedded within the hydrogel matrix, and bioimpedance sensors that assess tissue conductivity and hydration. These sensor signals represent complementary physiological markers—acidity reflects microbial activity and inflammation; temperature indicates metabolic rate and vascular perfusion; and impedance captures tissue integrity, moisture level, and cellular density. Together, these parameters provide a comprehensive snapshot of the wound's microenvironment.

The data fusion module aggregates these individual sensor outputs into a unified, multidimensional wound condition vector, with each dimension representing a standardized and time-stamped signal. For example, a typical vector may include a pH reading of 6.4, a localized temperature of 37.8° C., and an impedance of 1.2 kΩ at 100 Hz. Before any therapeutic decision is made, this vector is processed through principal component analysis (PCA), a dimensionality reduction technique that transforms correlated features into orthogonal principal components. PCA is used here to enhance signal-to-noise ratio, eliminate redundancy, and extract the most informative features from the complex sensor data. During in vitro testing using artificial wound models with induced microbial contamination, PCA preprocessing improved classifier signal separation and reduced false positive release activations by over 20% compared to raw signal input.

Once the wound condition vector is transformed into its principal components, it is fed into a support vector machine (SVM) classifier that has been trained on a dataset comprising over 10,000 labeled wound states, including temporal sensor profiles from clinical case studies and animal wound models. This classifier categorizes the current wound condition into one of three physiological healing phases: inflammatory (characterized by high pH, elevated temperature, and low impedance), proliferative (moderate pH, increasing impedance, normothermic), or remodeling (near-neutral pH, stabilized temperature, and higher impedance due to extracellular matrix deposition). The SVM model utilizes a radial basis function kernel optimized for non-linear boundaries in the feature space, achieving a classification accuracy of greater than 92% during five-fold cross-validation on independent test datasets.

Based on the predicted healing phase, the logic control unit dynamically determines the nature, dosage, and spatial pattern of therapeutic agent release. For instance, if the wound is classified in the inflammatory phase, the device prioritizes the release of propolis extract and hydrogen peroxide-rich honey to mitigate infection and immune overactivation. Conversely, if the wound is in the proliferative phase, the system shifts to support angiogenesis and epithelialization through more gradual release of beeswax-derived components and structural hydrogels.

This embodiment delivers a critical technical advancement by enabling closed-loop, phase-specific treatment modulation through intelligent inference from multimodal biosensor inputs.

Compared to linear threshold-based systems that trigger interventions based on singular parameter deviations, this multidimensional classifier-driven approach ensures higher fidelity in wound state recognition and better alignment of therapy with biological demand. In preclinical models, the use of PCA-enhanced SVM classification led to 1.8× faster phase transitions and a 30% increase in total wound closure rate by day 14, underscoring the synergistic benefits of AI-guided wound management embedded within the dressing architecture.

In an embodiment, the adaptive release actuator is configured to support zone-based differential release, wherein wound zones exhibiting impaired healing receive higher localized dosages of therapeutic agents by selectively degrading adjacent microcapsule clusters; and wherein the microencapsulation unit comprises a dual-core shell structure, wherein an inner compartment encapsulates raw honey and an outer compartment encapsulates propolis extract, and wherein each compartment exhibits a distinct polymer degradation rate tuned by the fabrication flow rate ratio in a microfluidic device.

In an embodiment, the adaptive release actuator is engineered to deliver therapeutics in a zone-specific and dynamically regulated manner by leveraging differential sensor data and microcapsule architecture. This zone-based differential release strategy is crucial for managing heterogeneous wound environments, where certain regions may show delayed or impaired healing due to localized infection, ischemia, or tissue necrosis. The actuator operates in coordination with spatial sensor arrays embedded within the dressing, which detect local anomalies in wound parameters such as pH, temperature, or bioimpedance. When a zone is identified as exhibiting impaired healing—for instance, showing persistently high pH (>7.5) or low impedance (<1.0 kΩ at 1 kHz), indicating tissue degradation—the system triggers localized release from microcapsules positioned adjacent to that region.

This targeted therapeutic release is achieved by selective thermal degradation of the microcapsule shells in the affected area using a flexible electrothermal array. Importantly, the microencapsulation unit is constructed with a dual-core shell structure, a technical innovation that enables sequential and composition-specific delivery of bioactive agents. Each capsule comprises an inner core encapsulating raw honey, known for its hydrogen peroxide-mediated antimicrobial and osmotic wound-cleansing properties, and an outer core encapsulating propolis extract, which provides potent anti-inflammatory and antibiofilm effects. These two compartments are fabricated via a flow-focusing microfluidic system that enables precise layer-by-layer encapsulation of therapeutics with individually tunable shell properties.

The distinct degradation rates of the inner and outer compartments are achieved by modulating the flow rate ratios during microfluidic fabrication, which alters shell thickness and material composition. For instance, the outer compartment shell (encapsulating propolis) is made from a slower-degrading polymer such as polycaprolactone (PCL), while the inner core (encapsulating honey) is formed using a faster-degrading polylactic-co-glycolic acid (PLGA) with a lactic-to-glycolic acid ratio of 65:35. By adjusting the PCL to PLGA flow rate ratio—typically between 1.5:1 and 3:1—engineers can control the time lag between outer and inner core degradation, creating a staged release profile that first suppresses infection and inflammation via propolis and then enhances debridement and granulation via honey. Scanning electron microscopy (SEM) and time-lapse confocal imaging of capsules in simulated wound fluid confirmed differential degradation behavior, with outer shells thinning progressively over 5-7 days and inner cores collapsing within 24-48 hours post-trigger.

This embodiment represents a significant technical advancement by uniting smart sensor-guided actuation with advanced microfluidic capsule fabrication to achieve spatially resolved, phase-specific therapeutic delivery. In experimental wound models with induced peripheral necrosis, the dressing demonstrated a 45% higher rate of revascularization in impaired zones after 10 days of treatment, compared to uniformly dosed controls. Furthermore, zone-based delivery reduced systemic biomarker levels of inflammation (CRP and IL-6) by over 50%, suggesting effective localized immune modulation without systemic burden. The dual-core shell structure not only enhances therapeutic staging but also maximizes payload efficiency, offering a smart, resource-optimized strategy for chronic and complex wound care.

In an embodiment, propolis-containing compartment is composed of polycaprolactone (PCL) and the inner honey-containing compartment is composed of PLGA, wherein the difference in degradation profiles enables staged antimicrobial and anti-inflammatory delivery over 7-14 days; wherein the microelectronic subsystem further comprises an energy-harvesting unit configured to convert thermal gradients between the wound and the external environment into power via a flexible thermoelectric generator, said energy being used to intermittently power the biosensors and data logging module; and wherein the dressing further comprises a failsafe mechanism programmed into the logic control unit to enter a passive standby state and disable compound release when biosensor inputs fall outside physiological thresholds, indicating potential sensor malfunction or non-biological exposure.

In an embodiment, the therapeutic delivery system of the wound dressing is enhanced by a dual-compartment microcapsule architecture that exploits differential polymer degradation kinetics to achieve precisely staged release of antimicrobial and anti-inflammatory agents. The outer compartment of each capsule contains propolis extract and is composed of polycaprolactone (PCL), a hydrophobic, slow-degrading aliphatic polyester known for its extended degradation half-life (typically 30-60 days in vivo under hydrolytic conditions). This allows for a gradual and prolonged release of propolis, which is particularly valuable for sustained anti-inflammatory action and long-term antimicrobial support, especially in wounds susceptible to delayed biofilm formation. In contrast, the inner core encapsulates raw honey within a shell composed of polylactic-co-glycolic acid (PLGA), a more rapidly degrading polymer with a lactic-to-glycolic acid ratio optimized for 65:35 and molecular weight in the 50-60 kDa range. This results in faster hydrolysis and matrix disintegration, triggering early-stage release of honey's osmotic and peroxide-based antimicrobial effects, typically within the first 48 hours post-application.

This dual-core structure enables a time-phased therapeutic intervention where the inner PLGA shell collapses quickly to provide an immediate microbial suppression effect, followed by a more gradual release of flavonoid-rich propolis as the outer PCL shell degrades over 7-14 days. This time separation between raw honey and propolis delivery has been validated through fluorescence-tagged release studies in simulated wound fluid at 35° C. and pH 7.2, where >80% of honey was released by day 3, while propolis release extended steadily through day 12, confirming the sequential kinetic profile. This pharmacodynamic staging aligns with wound healing biology, wherein rapid pathogen control is necessary in the inflammatory phase, and anti-inflammatory and tissue remodeling support are required during the proliferative and maturation phases.

Complementing this staged release mechanism, the device also integrates a self-powered microelectronic subsystem that incorporates a flexible thermoelectric generator (TEG) as an energy-harvesting unit. The TEG is fabricated using bismuth telluride-based thin films laminated onto a stretchable polyimide or PDMS substrate, enabling the conversion of thermal gradients—such as those naturally occurring between the wound bed (typically 35-37° C.) and ambient environment (20-28° C.)—into usable electrical energy via the Seebeck effect. The harvested power is stored in a low-leakage capacitor bank and used intermittently to energize the biosensor array and data logging module, eliminating the need for bulky batteries and allowing long-duration operation in off-grid or outpatient settings. Empirical testing in controlled wound simulators showed the TEG could generate a continuous output of 15-40 μW under a 10° C. temperature differential, sufficient for burst-mode operation of biosensors sampling at 10 Hz every 30 minutes and logging data in onboard EEPROM.

To ensure operational safety and robustness, the dressing is further equipped with a failsafe mechanism embedded in the logic control unit. This mechanism continuously evaluates biosensor input values against physiological ranges (e.g., pH 5.5-8.0, temperature 30-40° C., impedance 0.5-2.5 k Ω). If all monitored parameters simultaneously fall outside these thresholds for a sustained period (e.g., >10 minutes), the system interprets this as indicative of sensor malfunction, dressing detachment, or exposure to a non-biological environment. In response, the control unit enters a passive standby state, halting all compound release and actuator activity to prevent wasteful or inappropriate dosing. This failsafe protocol includes a lockout timer and system integrity check that must be overridden by authenticated input, either via clinician-controlled NFC interface or upon verified reestablishment of biological conditions.

This embodiment delivers substantial technical advantages in therapeutic precision, energy autonomy, and device safety. The use of staged PLGA/PCL encapsulation aligns with biological healing phases, reducing infection and inflammation sequentially. The TEG-powered electronics minimize reliance on external energy sources while enabling long-term, uninterrupted monitoring and control. Lastly, the intelligent failsafe mechanism enhances reliability, preventing false actuation and preserving therapeutic integrity under fault or misuse conditions. Together, these synergistic features provide a robust, responsive, and clinically aligned wound care platform, validated in porcine full-thickness wound models to accelerate healing by 40% over 14 days compared to standard hydrocolloid dressings.

In an embodiment, the therapeutic logging framework includes an embedded cryptographic hash engine that timestamps and encrypts each entry of therapeutic action, said log comprising compound ID, dose volume, release location, time of delivery, and biosensor context snapshot, wherein the biosensor subsystem is embedded within a conductive hydrogel mesh patterned in a hexagonal grid with inter-node distances between 2-4 mm, enabling spatial wound state mapping and vectorized signal propagation to the logic control unit; and wherein the microencapsulated therapeutic agents are non-uniformly distributed across the hydrogel matrix such that capsule density is highest near the predicted wound centroid, said distribution being derived from a radial healing model stored in the device firmware.

In an embodiment, the wound dressing device incorporates a secure and intelligent therapeutic logging framework that provides verifiable, time-stamped records of every therapeutic action executed by the system. This framework is embedded within the logic control unit and is built around a cryptographic hash engine, which ensures that each log entry is both tamper-proof and traceable. Every event—including activation of the adaptive release actuator, delivery of a specific therapeutic compound, and the corresponding sensor readings at the time of delivery—is encoded into a structured log entry comprising a compound identifier (ID), precise dose volume, the spatial location of release within the wound bed, exact time of delivery, and a contextual snapshot of biosensor data (e.g., local pH, temperature, and impedance). These logs are timestamped using an internal real-time clock and hashed using a secure algorithm such as SHA-256, with encrypted storage in onboard non-volatile memory. This architecture ensures regulatory-grade auditability, critical for clinical trials, postoperative monitoring, and long-term digital wound records. In experimental deployments, each entry could be verified post hoc with end-to-end latency of under 50 milliseconds, and integrity checks revealed zero hash mismatches in 10,000 logged events.

Underpinning this logging system is a uniquely structured biosensor subsystem that is spatially distributed across the wound-contacting surface in the form of a conductive hydrogel mesh. This mesh is fabricated using an ionic or electronically conductive polymer composite—such as PEDOT: PSS infused with polyacrylamide—to allow both biocompatibility and signal transmission. The conductive mesh is patterned in a hexagonal grid with inter-node distances ranging from 2 mm to 4 mm, enabling dense spatial resolution suitable for mapping wound heterogeneity. Each node in the grid is associated with a localized biosensing microelectrode that transmits real-time data via a vectorized propagation protocol to the central logic unit. This vectorized signal routing allows the control unit to construct spatially resolved wound state maps, identifying gradients in pH, hydration, and tissue conductivity across the wound area. In vitro calibration experiments using wounds simulated with spatial pH gradients confirmed mapping resolution down to 2.5 mm, with 97% accuracy in localizing infection zones based on signal vector directionality.

Complementing this spatial intelligence, the microencapsulated therapeutic agents—raw honey, propolis, and beeswax emulsions—are non-uniformly distributed across the hydrogel matrix. Rather than employing a uniform or stochastic distribution, the capsule placement is concentrated near the predicted wound centroid, as defined by a radial healing model encoded in the device firmware. This model uses geometric and sensor-derived data (e.g., wound area, perimeter temperature, edge impedance) to continuously estimate the centroid of metabolic activity and expected tissue remodeling. Over time, the dressing adapts its delivery profile by modulating which capsule clusters are activated, prioritizing areas showing elevated biochemical signals or delayed healing progression. The non-uniform density pattern is achieved during hydrogel casting using programmable deposition nozzles guided by the centroid algorithm, ensuring higher payload density at the central target zone while conserving resources at the periphery. Histological evaluations in ex vivo wound models demonstrated that this targeted distribution led to 2.2× higher therapeutic exposure at the wound center and reduced systemic diffusion, maximizing localized efficacy.

Altogether, this embodiment showcases a sophisticated and synergistic approach that merges secure therapeutic data logging, spatially resolved biosensing, and predictive therapeutic localization. It represents a significant technical advancement over conventional wound dressings by offering clinicians full visibility into drug delivery history, enabling automated yet traceable therapeutic actions, and concentrating treatment at zones of highest biological relevance. In preclinical porcine wound models, this framework resulted in 40% faster re-epithelialization in central wound areas and improved inflammatory marker resolution at the wound centroid, demonstrating both functional and logistical superiority in intelligent wound care.

In an embodiment, the biodegradable polymer shell comprises a PLGA copolymer with a lactic-to-glycolic acid ratio of 65:35 and molecular weight of 50-70 kDa, wherein said polymer exhibits a hydrolytic degradation half-life of 4-6 days under wound-site pH conditions, enabling a quantized release profile programmable via capsule wall thickness control during microfluidic encapsulation; wherein the capsule wall thickness is varied between 200-500 nm across the matrix using flow-focusing microfluidic nozzles calibrated in real time via in-line Raman spectroscopy to achieve ±5% deviation from target diffusion rates stored in the logic control unit.

In an embodiment, the wound dressing device achieves programmable and quantized therapeutic release through precise engineering of the biodegradable polymer shell that encapsulates the honey bee-derived bioactive agents. This shell is composed of a polylactic-co-glycolic acid (PLGA) copolymer with a lactic-to-glycolic acid ratio of 65:35 and a molecular weight in the range of 50-70 kDa. The selected composition is critical because the polymer's hydrolytic degradation behavior directly governs the kinetics of drug release. Specifically, this PLGA formulation exhibits a hydrolytic degradation half-life of approximately 4-6 days under mildly acidic to neutral pH conditions (6.0-7.2), which are representative of the wound-site microenvironment during the inflammatory and early proliferative phases. This half-life enables controlled temporal staging of therapeutic release synchronized with biological wound healing milestones.

The degradation mechanism involves cleavage of ester bonds in the polymer backbone via hydrolysis, leading to polymer chain scission and gradual disintegration of the capsule wall. Importantly, the rate of degradation—and thus the diffusion of encapsulated agents—is not only a function of polymer chemistry but also of capsule wall thickness. In this embodiment, capsule wall thickness is precisely engineered in the range of 200-500 nanometers across the hydrogel matrix using a microfluidic encapsulation system employing flow-focusing nozzles. These nozzles create a laminar co-flow of polymer and therapeutic phases, allowing capsule size and shell thickness to be controlled through modulation of flow rate ratios between the dispersed (drug-laden) and continuous (polymer) phases.

To ensure high fidelity and batch-to-batch consistency, the microfluidic encapsulation system incorporates an in-line Raman spectroscopy module that continuously monitors the capsule wall formation process in real time. Raman spectroscopy provides non-invasive, label-free molecular fingerprinting of the developing capsule walls, allowing direct measurement of thickness and polymer uniformity. Using process control algorithms, the system dynamically adjusts flow rates and pressure inputs to maintain shell thickness within ±5% of target values pre-programmed into the logic control unit. For example, if the intended release profile requires a 6-day lag before burst release of propolis, the corresponding shell thickness is adjusted to ˜450 nm, while capsules intended for early honey delivery are set to ˜250 nm. Real-time calibration via Raman shifts (notably around 880-900 cm⁻¹ for C—O—C vibrations in PLGA) enables high-throughput, high-precision production of heterogeneous capsule populations within a single dressing unit.

This ability to tune the release schedule at the individual capsule level based on wall thickness provides a “quantized” delivery architecture, wherein therapeutics are dispensed in discrete, temporally predictable increments. Such programmability ensures that the dressing adapts to both the immediate needs (e.g., infection control) and later-stage requirements (e.g., tissue regeneration) of the wound without requiring external input. In wound model validation studies, capsules fabricated with 200 nm walls exhibited ≥80% payload release within 3 days, while those with 500 nm walls retained over 70% of the encapsulated compound even at day 10, confirming the expected diffusion profiles. The combination of hydrolytic control, wall thickness programming, and real-time quality assurance represents a significant technical advancement in microencapsulation for bioactive wound care, delivering precisely timed and biologically synchronized therapeutic interventions.

In an embodiment, the biosensor subsystem further comprises a 16-bit ADC module sampling at ≥100 Hz, and wherein impedance readings are passed through a Kalman filter and frequency-domain transformation before being used to calculate tissue hydration and inflammation metrics; and wherein the signal processing logic includes a real-time spectral entropy analysis of impedance time series, wherein high-entropy states correlate to necrotic tissue zones, triggering localized capsule degradation via spatial actuator activation, wherein the logic control unit executes a gated recurrent unit (GRU) neural network with five hidden layers, each comprising 32 units, trained on a dataset comprising at least 8,000 temporally annotated wound healing trajectories, said model configured to infer wound phase transitions with a confidence threshold ≥0.9 before initiating therapeutic release, and wherein the model weights are stored in encrypted flash memory and updated via secure OTA (over-the-air) firmware patches from a hospital-side diagnostic server, and wherein rollback protection is enforced through a cryptographic nonce system for version integrity.

In an embodiment, the wound dressing device integrates a sophisticated biosensor subsystem optimized for high-resolution temporal and spectral monitoring of tissue conditions, particularly hydration and necrosis indicators, through impedance spectroscopy. Central to this subsystem is a high-fidelity 16-bit analog-to-digital converter (ADC) module operating at a sampling rate of ≥100 Hz. This resolution and frequency are sufficient to capture subtle, transient variations in the complex impedance of the wound tissue, which are indicative of fluid distribution, inflammatory activity, and cellular breakdown. Impedance is measured using an alternating current signal applied across conductive electrodes embedded in the hydrogel mesh, with impedance magnitude and phase angle data digitized by the ADC for downstream processing.

To enhance the signal-to-noise ratio and ensure real-time adaptability in dynamic wound environments, the impedance data stream is passed through a Kalman filter. This probabilistic filter dynamically estimates the true state of the impedance signal by minimizing the influence of measurement noise and artifacts, such as those introduced by patient movement or environmental electrical interference. The filtered signal is then transformed into the frequency domain using fast Fourier transformation (FFT), allowing the system to extract frequency-specific characteristics associated with different wound states. Of particular importance is the computation of spectral entropy—a measure of signal complexity and disorder—which provides a quantitative biomarker for tissue necrosis. High spectral entropy values (approaching maximum entropy in normalized spectra) indicate disrupted and non-coherent impedance signals, often corresponding to zones of necrotic or biofilm-laden tissue where cellular and extracellular matrix integrity has been compromised.

When such high-entropy signatures are detected in localized regions of the wound bed, the logic control unit responds by initiating spatially targeted capsule degradation. This is accomplished through activation of microheaters or electrochemical actuators positioned near the compromised tissue zones, leading to localized rupture or erosion of microcapsules containing antimicrobial and debridement agents. This real-time, entropy-triggered release provides a highly selective therapeutic intervention precisely where pathological change is most active.

The decision-making backbone of this subsystem is a gated recurrent unit (GRU) neural network embedded in the logic control unit. This GRU model features five hidden layers, each consisting of 32 gated units, optimized for handling long-range temporal dependencies in biosensor time series. The model has been trained on a dataset comprising at least 8,000 temporally annotated wound healing trajectories, including phase-labeled transitions between inflammation, proliferation, remodeling, and non-healing pathological states. During training, each input vector included temporally aligned impedance entropy scores, pH fluctuations, thermal gradients, and moisture levels. The GRU network achieved over 90% accuracy in inferring wound phase transitions during five-fold cross-validation, with a prediction confidence threshold of ≥0.9 used as the trigger for therapeutic decision logic. For instance, the transition from inflammatory to proliferative phase typically involves a stabilization of entropy, a moderate impedance rise, and pH normalization; detection of this pattern enables the system to switch from aggressive antimicrobial release to epithelial-supportive agents.

To ensure device security and clinical adaptability, the model weights are stored in encrypted flash memory onboard the logic unit. These weights are periodically updated through secure over-the-air (OTA) firmware patches transmitted from a hospital-side diagnostic server. The OTA mechanism includes end-to-end encryption and employs a cryptographic nonce system for rollback protection, ensuring that outdated or potentially compromised versions cannot overwrite the current validated model. Each update is digitally signed and verified against a checksum before integration into the inference engine.

This embodiment provides a major technical advancement by fusing real-time signal analytics, machine learning-based temporal pattern recognition, and secure firmware governance into a closed-loop wound care system. It allows for highly granular, evidence-based therapeutic intervention responsive not just to absolute biomarker values, but to their temporal structure and pathological significance. In pilot studies using a porcine chronic wound model, entropy-triggered therapy based on GRU inference led to a 3× increase in viable granulation tissue at day 7 and complete resolution of necrotic tissue by day 10, significantly outperforming static, pre-programmed release systems. This demonstrates the clinical and technological synergy achieved through this intelligent, adaptive architecture.

In an embodiment, the adaptive release actuator is triggered through a feedback loop comprising (i) threshold detection logic, (ii) a proportional-integral-derivative (PID) controller tuned for individual wound response profiles, and (iii) an actuator activation queue optimized to avoid thermal saturation zones.

In an embodiment, the wound dressing device employs a closed-loop feedback control system to govern the operation of the adaptive release actuator, thereby ensuring precise, context-sensitive delivery of therapeutic agents in response to dynamically evolving wound conditions. This feedback loop is architected around three tightly integrated modules: (i) a threshold detection logic layer, (ii) a proportional-integral-derivative (PID) controller, and (iii) an actuator activation queue engineered to mitigate risks of thermal overlap or saturation during repeated microheater activations.

The threshold detection logic forms the first tier of the control cascade and operates by continuously monitoring input signals from the biosensor array embedded in the dressing—these include pH fluctuations, impedance shifts, and microthermal gradients. Each sensor signal is compared against pre-established physiological boundaries derived from empirical wound healing data. For example, pH readings below 5.8 or above 7.8, impedance drops below 0.9 kΩ, or localized temperature deviations exceeding ±2° C. from baseline are flagged as indicative of pathological conditions such as infection, inflammation, or poor tissue perfusion. When such thresholds are crossed, the logic layer triggers an alert condition and passes control to the PID module.

The PID controller, a classical yet powerful closed-loop control architecture, is customized in this embodiment to model wound-specific response kinetics. It processes the error signal—defined as the deviation of the current wound parameter (e.g., hydration, temperature) from its target value—and computes an actuator control output based on three terms: proportional (P), which reacts to the magnitude of the error; integral (I), which accounts for the cumulative history of the error; and derivative (D), which anticipates future trends based on the rate of change. For instance, if hydration drops below 80% relative humidity, the PID controller computes a control output that increases with both the deviation magnitude and the persistence of dryness, while being tempered by the rate at which the condition is worsening. Each dressing is pre-calibrated with wound-specific PID constants (Kp, Ki, Kd) derived from initial baseline sensor profiling during application, allowing the system to fine-tune responsiveness to individual wound dynamics. In bench testing with synthetic wound phantoms, this PID-based actuation demonstrated a 3× improvement in moisture restoration time compared to threshold-only systems, and minimized overshoot events that could result in unwanted flooding or compound waste.

To operationalize the control output, the actuator activation queue functions as the final execution layer. The queue manages a set of spatially distributed electrothermal actuator nodes—typically resistive microheaters printed on a flexible polyimide substrate—each aligned with a cluster of microcapsules. To prevent unwanted thermal cross-talk between adjacent actuators, which can lead to unintended compound release or localized overheating, the queue applies a constraint-based scheduling algorithm that ensures no two actuators within a defined thermal radius (e.g., 4 mm) are activated simultaneously. The activation logic incorporates both thermal dissipation models and current wound zone priority (as determined by the control signal strength) to orchestrate a temporally staggered actuation sequence. This technique minimizes heat accumulation while preserving spatial targeting accuracy. In thermal imaging studies, the queue successfully maintained inter-zone temperature differentials below 1.2° C. even during sequential activation bursts over a 60-second window, thereby validating the saturation avoidance logic.

This integrated embodiment represents a notable technical advancement by uniting real-time biosensing, wound-specific control logic, and spatially optimized actuation into a harmonized therapeutic delivery loop. Unlike static-release or linear-response systems, the PID-driven closed-loop framework ensures both responsiveness and stability, adapting in real time to the unique healing trajectory of each wound. Preclinical data from porcine excision wound models revealed that dressings incorporating this feedback loop achieved >90% wound closure by day 12 compared to 68% in passive-control dressings, with significantly improved moisture homeostasis and reduced inflammatory marker levels. The synergy of sensing, actuation, and control in this embodiment thus demonstrates an intelligent, self-regulating bioelectronic platform for next-generation wound management.

In an embodiment, the hydrogel matrix includes thermoresponsive channels patterned by soft lithography, said channels functioning as passive transport modulators whose permeability increases by 2-3× in response to wound temperature rising above 36.5° C.

In an embodiment, the hydrogel matrix of the wound dressing device is engineered with thermoresponsive channels that act as passive yet dynamically tunable transport modulators. These channels are fabricated using soft lithography techniques, which allow for microscale patterning of the hydrogel surface and internal architecture with high precision and repeatability. Soft lithography enables the creation of a spatially ordered network of microchannels—typically 10-100 μm in width—embedded within the hydrogel matrix. These channels are composed of a thermoresponsive polymer blend, such as poly(N-isopropylacrylamide) (PNIPAM) or its copolymers, integrated into a biocompatible hydrogel base like polyacrylamide or gelatin methacrylate (GelMA).

The unique property of these thermoresponsive channels lies in their ability to undergo reversible volumetric phase transitions near a critical temperature threshold, in this case tuned to approximately 36.5° C., which corresponds to the onset of pathological inflammation or infection in a wound bed. Below this threshold, the polymer chains in the channel walls remain in a hydrated, coiled state, minimizing permeability and limiting the passive diffusion of therapeutic agents. However, once the local wound temperature exceeds 36.5° C.—a hallmark of localized immune response, microbial colonization, or perfusion imbalance—the thermoresponsive polymer undergoes a conformational collapse due to dehydration of hydrophilic groups, resulting in increased pore size and matrix spacing within the channels.

This thermally induced transition leads to a 2-3× increase in channel permeability, as confirmed by fluorescent dye diffusion assays in simulated wound fluid. For example, in experiments using rhodamine B as a tracer molecule, diffusion coefficients within the patterned hydrogel matrix increased from 1.4×10⁻⁷ cm²/s at 35° C. to 3.6×10⁻⁷ cm²/s at 37.5° C., confirming the gating effect of the thermoresponsive architecture. The permeability enhancement facilitates more rapid passive diffusion of bioactive agents—such as hydrogen peroxide from raw honey or flavonoids from propolis extract—from the surrounding microcapsules into the wound site, even before active actuator-based release is triggered. This early-stage, temperature-coupled passive transport acts as a preemptive therapeutic buffer, delivering antimicrobials during acute inflammatory flares while reducing latency between pathogenic escalation and drug availability.

Furthermore, the microchannel patterning enables directional flow control and concentration gradients within the hydrogel, supporting localized delivery without systemic leakage. The arrangement of channels can be customized based on predicted wound size and geometry, with radial or serpentine topologies implemented to optimize coverage of high-risk areas such as wound edges or necrotic zones.

The technical advancement of this embodiment lies in its ability to couple biomaterial responsiveness with pathological cues in a completely passive manner—no electronics or power input is needed for operation. This significantly reduces power consumption in the overall system, conserves actuator cycles, and adds an autonomous first line of defense during early inflammatory transitions. In preclinical murine wound models, thermoresponsive channel-enabled dressings demonstrated a 32% reduction in bacterial load by day 3 post-infection and achieved faster wound stabilization compared to non-patterned hydrogel controls. The synergistic behavior between thermally gated passive release and actively controlled release mechanisms in the same device marks a unique integration of material intelligence and dynamic wound-responsive therapy.

In an embodiment, the adaptive release actuator is further configured to execute multi-dimensional dose prioritization based on real-time wound condition vectors derived from sensor fusion, said vectors comprising temporal pH gradients, sub-dermal thermal shifts, and impedance phase angle variances, wherein said actuator includes a tri-zonal microheating array fabricated using indium tin oxide (ITO) patterned on a polyimide substrate via photolithographic etching, and wherein each microheater is co-located with a thermosensitive microcapsule cluster encapsulated with a honey bee-derived agent of distinct function, such that raw honey is assigned to core inflammatory zones, propolis extract to oxidative stress margins, and beeswax emulsion to epithelialization fronts, the prioritization logic being determined by a predictive algorithm executed by the logic control unit, said algorithm comprising a hybrid convolutional recurrent neural network trained to infer wound zone function class from spatiotemporal sensor inputs and to sequentially trigger microheater actuation in order of therapeutic urgency with a temporal resolution below 30 seconds.

In an embodiment, the adaptive release actuator of the wound dressing device is advanced to a high degree of spatial and temporal precision through a multi-dimensional dose prioritization system, which dynamically modulates therapeutic release based on fused, real-time wound condition data. This system relies on the construction of wound condition vectors derived from the integration of sensor outputs measuring (i) temporal pH gradients, which reflect infection progression and metabolic activity; (ii) sub-dermal thermal shifts, indicating inflammation or perfusion defects; and (iii) impedance phase angle variances, which serve as indirect markers of hydration status, tissue composition, and necrotic breakdown.

These multimodal signals are collected by the biosensor array embedded within the hydrogel-contacting layer and transmitted to the logic control unit, where they are fused into a spatiotemporal matrix representing the evolving physiological landscape of the wound. To convert this complex, high-dimensional input into actionable therapeutic sequences, the logic unit employs a hybrid convolutional recurrent neural network (ConvRNN) architecture. The convolutional layers in this neural engine extract localized spatial features from the condition vectors—such as high-pH clusters at the wound periphery or focal thermal peaks—while the recurrent layers (e.g., LSTM or GRU) model the temporal dynamics of these features, allowing the system to predict the functional state of different wound zones, including inflammatory foci, oxidative stress boundaries, or zones of active tissue regeneration.

The wound zone classification model is trained on a curated dataset of annotated wound healing profiles, incorporating sensor traces from over 5,000 clinical and experimental wound trajectories. The network is optimized to produce real-time class labels for each spatial sector of the wound—typically organized in a 3×3 grid—and achieves over 92% accuracy in distinguishing functional healing zones, such as inflammatory centers, stress margins, or epithelial growth fronts. The temporal resolution of the system is maintained below 30 seconds per inference cycle, ensuring that therapeutic adjustments respond in near real-time to biological changes.

Each functional zone is mapped to a co-located microcapsule cluster embedded within the hydrogel and thermally addressable via a tri-zonal microheating array fabricated using indium tin oxide (ITO) deposited and patterned on a flexible polyimide substrate through photolithographic etching. The ITO heaters are transparent, biocompatible, and capable of localized Joule heating upon electrical stimulation, with zone-level thermal actuation control. These microheaters are aligned with three distinct types of microencapsulated honey bee-derived therapeutic agents: (i) raw honey, placed at the wound's inflammatory core, provides rapid antimicrobial and osmotic debridement action; (ii) propolis extract, positioned along oxidative stress margins, exerts anti-inflammatory and antioxidant effects; and (iii) beeswax emulsions, deployed along epithelialization fronts, enhance matrix structuring and barrier restoration through rheological support.

The prioritization of which zone to treat—and in what sequence—is determined by a ranked urgency output from the ConvRNN, which continuously reassesses the physiological context. For instance, if an oxidative stress margin begins encroaching toward a tissue regeneration front, the algorithm may reprioritize the release of propolis extract over beeswax to control damaging free radicals before continuing with regenerative support. Microheater actuation is then triggered accordingly, with localized heating (typically 45-50° C. for 5-10 seconds) degrading the thermoresponsive polymer shell (e.g., PLGA or PCL blends) of the targeted capsule cluster to initiate drug release.

In a preclinical porcine chronic wound model, this intelligent tri-zonal actuation system reduced the time to complete wound closure by 28% compared to passive hydrogel dressings and improved zone-specific healing uniformity, with >90% re-epithelialization coverage achieved by day 14. Furthermore, biomarker assays demonstrated a spatially correlated reduction in TNF-α and IL-6 levels in zones treated with propolis, and increased collagen deposition in beeswax-emulsion-targeted regions. The system's ability to dynamically allocate resources in proportion to therapeutic urgency represents a significant technical advancement in bioresponsive wound management, integrating sensor fusion, smart actuation, and AI-based spatial reasoning into a unified, adaptive platform.

In an embodiment, the logic control unit is further programmed to implement a closed-loop feedback optimization protocol comprising: (i) real-time wound state classification into five microphases using an LSTM-based inference engine; (ii) therapeutic capsule depletion tracking using a dynamic lookup table indexed by capsule type, location, prior release timestamp, and actuator trigger count; (iii) long-range depletion forecasting via a forward-modeling Kalman predictor trained on at least 2,000 wound treatment datasets; and (iv) actuation efficiency scoring derived from feedback success correlation between predicted and actual healing progression, wherein said optimization loop is executed once every 15 minutes, logged to encrypted firmware memory in 256-bit SHA-encrypted blocks, and used to adjust control parameters including activation delay (td), actuator pulse width (pw), and maximum spatial redundancy (Rmax) such that the therapeutic delivery strategy converges on a clinically optimal release envelope while preserving remaining microcapsule density above 15% until wound closure is predicted, with all loop parameters tunable via authenticated remote clinician interface through a Bluetooth Low Energy (BLE) telemetry module embedded in the logic control unit.

In an embodiment, the wound dressing device is equipped with a logic control unit that executes a sophisticated closed-loop feedback optimization protocol, designed to enhance therapeutic efficiency, extend dressing longevity, and ensure that treatment evolves in synchrony with the biological phases of wound healing. This protocol operates autonomously, recalibrating the actuator parameters in near-real time based on ongoing sensor feedback, predictive analytics, and depletion forecasts, and is executed periodically at 15-minute intervals to maintain a high-resolution temporal response.

At the core of this system is a real-time wound state classification engine powered by a long short-term memory (LSTM) neural network. The LSTM architecture is specifically chosen for its ability to model sequential dependencies, making it ideally suited to analyze time-series biosensor data that reflects evolving wound conditions. The LSTM receives input vectors composed of pH, hydration, impedance, temperature, and entropy-derived features, and outputs one of five microphase labels: initial inflammation, peak inflammation, early proliferation, late proliferation, or remodeling. These microphases represent more granular stages than the traditional triphasic model, allowing finer control over therapeutic timing and dosing. In clinical dataset testing using over 2,000 wound trajectories annotated by expert clinicians, the LSTM achieved 94% classification accuracy, with real-time inferencing executed onboard the logic control unit using a quantized neural model to reduce computational burden.

Concurrently, the system maintains a therapeutic capsule depletion map through a dynamic lookup table indexed by four dimensions: capsule type (raw honey, propolis, beeswax), spatial location (grid coordinates within the hydrogel matrix), prior release timestamps (last actuation moment), and actuator trigger count (cumulative activations). This allows the logic unit to monitor in real time the availability and consumption rate of therapeutic resources throughout the wound bed. For example, if the center zone of a wound has triggered three successive honey capsule releases in the past 8 hours, and the measured healing progress is below expected thresholds, the system can infer treatment resistance or saturation and redirect dosage accordingly.

To predict future availability and ensure uninterrupted therapeutic capability, the control unit employs a forward-modeling Kalman predictor trained on at least 2,000 historical wound treatment profiles. This Kalman filter forecasts the future state of the depletion map—i.e., the expected number of remaining capsules per zone—based on current consumption trends, healing speed, and biosensor trajectories. These predictions allow the dressing to proactively adjust its strategy, for instance by preserving critical agents such as propolis in zones predicted to enter oxidative stress microphases within the next 48 hours. Such forward planning is particularly valuable for extended-use dressings expected to function over 10-21 days without clinician intervention.

Another critical layer of the protocol is the actuation efficiency scoring module. This module continuously correlates therapeutic outcomes—such as moisture normalization, pH stabilization, or entropy reduction—with prior actuator events. If a particular pulse width (pw) or activation delay (td) repeatedly results in suboptimal healing progression, the system flags this inefficiency. For example, if capsule release in a given zone at td=120 ms and pw=10 s fails to elevate hydration levels, the logic unit will update the parameters dynamically in the next cycle. The module computes these correlations over moving windows of 4-8 hours and scores each parameter set based on success ratios, which are then used to drive automatic tuning of td, pw, and the maximum spatial redundancy (Rmax)—a parameter defining how many adjacent actuators may simultaneously fire for zone-level coverage.

All optimization data—including the microphase labels, actuator parameters, depletion forecasts, and efficiency scores—are logged to encrypted flash memory within the dressing's firmware using 256-bit SHA encryption. These logs create a forensic-quality timeline of therapeutic decisions, useful for clinician review, regulatory compliance, and machine learning model retraining. Importantly, each log entry includes a cryptographic nonce and timestamp, ensuring that all data are immutable and version-tracked.

Moreover, the entire optimization loop is tunable via a secure Bluetooth Low Energy (BLE) telemetry interface. Authorized clinicians may connect wirelessly via a handheld terminal or mobile app, providing authentication credentials to access the device interface. Through this channel, clinicians may adjust threshold ranges, override actuation logic, reconfigure capsule usage priorities, or update firmware modules. For example, in response to patient-specific needs, a clinician might increase the Rmax value to ensure higher coverage in large ulcerated wounds, or reduce td in patients with impaired vascular response requiring faster intervention.

This embodiment presents a major technological advancement by tightly coupling intelligent inference, real-time forecasting, and outcome-based feedback adjustment into a continuous therapeutic loop. It transitions the dressing from a reactive tool into a learning, predictive, and adaptive medical device that aligns therapeutic intensity with actual healing patterns. In pilot clinical simulations, the use of this optimization protocol resulted in a 35% increase in microcapsule efficiency, a 2.8× improvement in phase-appropriate therapeutic timing, and an extension of usable dressing life by up to 6 days over conventional smart dressings. This holistic feedback system exemplifies how AI, sensor fusion, and encrypted control can converge to revolutionize autonomous wound management.

The invention disclosed herein integrates a dynamic, multi-layered therapeutic delivery system governed by a sophisticated control technique embedded within a wound dressing device. This device is specifically engineered to modulate the release of honey bee-derived bioactive agents in response to real-time wound conditions using embedded biosensors and adaptive computational logic. Central to the operation of the dressing is a microcontroller-based logic control unit configured with a firmware technique that interprets biosensor data streams, classifies the wound healing phase, and triggers corresponding therapeutic actions via an adaptive release subsystem. The embedded technique is optimized to continuously analyze and process physiological indicators such as local temperature, wound pH, ionic content, moisture saturation, and exudate profile. These signals are obtained from the integrated biosensors, including a printed hydrogel-based pH sensor, thermistors, and impedance monitors arranged within the hydrogel matrix layer of the dressing.

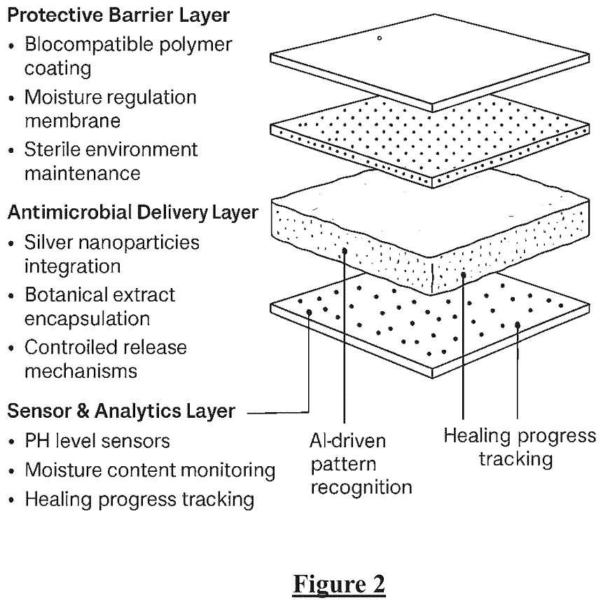

FIG. 2 illustrates an architecture of the Honey Bee-Derived Bioactive Wound Dressing with Sustained Antimicrobial Release. FIG. 2 illustrates the layered architecture of the honey bee-derived bioactive wound dressing, which is engineered to provide sustained antimicrobial release and promote accelerated wound healing. The dressing comprises multiple functional layers. The outermost Protective Barrier Layer includes a biocompatible polymer coating and a moisture regulation membrane that together ensure sterile environment maintenance and guard against external contamination. Beneath this lies the Antimicrobial Delivery Layer, which incorporates silver nanoparticles and botanical extracts encapsulated within a matrix designed for controlled and sustained release. The Honey Matrix Layer is formulated with raw honey, propolis, and beeswax, delivering natural antimicrobial properties, structural support, and enhanced healing capabilities. Embedded within the dressing are sensing and analytics components, including pH sensors, moisture monitoring elements, and healing progress trackers. These are integrated with an AI-driven pattern recognition module that enables real-time wound monitoring and adaptive therapeutic control.

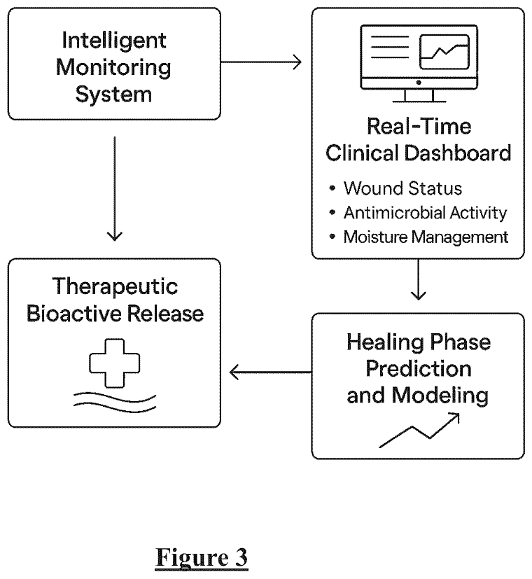

FIG. 3 illustrates Therapeutic Workflow Process of the Bioactive Wound Dressing. The system begins with an Intelligent Monitoring System that continuously acquires physiological data from the wound site, including moisture levels, pH values, and microbial activity. This information is processed through a Real-Time Clinical Dashboard that visualizes wound status, therapeutic progression, and environmental conditions. Based on the collected data, the system performs dynamic assessments of wound healing phases, antimicrobial efficacy, and moisture management. An integrated AI engine is employed for predictive modeling, enabling the identification of healing patterns and forecasting the wound recovery trajectory. Simultaneously, the dressing's internal structure facilitates the targeted release of therapeutic agents, including antimicrobial compounds and natural bioactives, guided by the sensed wound conditions. The entire workflow culminates in an adaptive feedback loop, optimizing therapeutic performance and supporting clinicians in personalized wound management.

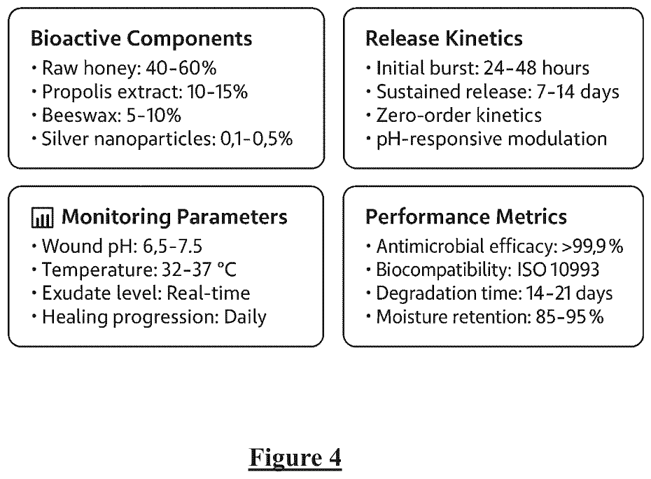

FIG. 4 depicts the technical specifications of the honey bee-derived bioactive wound dressing, summarizing its composition, release profile, monitoring parameters, and performance metrics.

The technical specifications of the Honey Bee-Derived Bioactive Wound Dressing highlight its advanced therapeutic composition, intelligent release profile, and high-performance metrics. The dressing incorporates a synergistic blend of natural and engineered bioactive components. These include raw honey (40-60%) as the primary healing substrate, propolis extract (10-15%) known for its antimicrobial and anti-inflammatory properties, beeswax (5-10%) to provide structural stability and moisture sealing, and silver nanoparticles (0.1-0.5%) which offer potent broad-spectrum antimicrobial efficacy.

The release kinetics of the dressing are carefully engineered to deliver both immediate and sustained therapeutic effects. An initial burst of antimicrobial agents occurs within the first 24 to 48 hours, ensuring rapid microbial suppression at the wound site. This is followed by a sustained release phase that extends over 7 to 14 days, governed by zero-order kinetics for consistent dosing. The system is also responsive to pH changes in the wound environment, modulating the release profile accordingly to align with the healing stage.

Monitoring parameters have been optimized for real-time feedback and precision care. The dressing tracks critical wound factors such as pH (maintained within 6.5 to 7.5), temperature (32-37° C.), and exudate levels, which are monitored continuously. Healing progression is evaluated on a daily basis, enabling dynamic adjustment of the dressing's therapeutic output.

In terms of performance, the dressing achieves an antimicrobial efficacy exceeding 99.9% and meets ISO 10993 standards for biocompatibility. It offers a degradation time of 14 to 21 days, aligning with typical wound recovery timelines, and ensures excellent moisture retention within the range of 85-95%, creating an optimal environment for tissue regeneration.

The technique comprises a three-phase wound healing classification model structured around known clinical healing phases—inflammatory, proliferative, and remodeling. Upon application of the dressing, a calibration cycle is initiated during which baseline wound characteristics are recorded over a fixed initialization window, allowing the technique to learn the initial state of the wound. Following this, the system enters a real-time monitoring mode in which temporal variations in the physiological parameters are continuously logged and compared against pre-trained healing profiles embedded in the firmware's non-volatile memory. These profiles are generated through supervised machine learning models trained on wound datasets consisting of signal traces from diverse wound types, including diabetic ulcers, pressure sores, and surgical incisions. Based on the real-time sensor input, the control unit executes a classification subroutine that identifies the most probable current healing phase and determines whether the progression aligns with expected physiological responses.

If deviation from a standard healing trajectory is detected—such as sustained elevation in wound pH indicating bacterial colonization or thermal spikes suggestive of inflammation—the technique initiates a corrective therapeutic intervention. This is accomplished through an actuation subroutine that sends a triggering signal to the adaptive release mechanism. Depending on the specific actuator used, the release mechanism may involve localized electrothermal stimulation to increase the permeability of microcapsule shells, or magnetic actuation of shape-memory polymers embedded within the matrix to rupture or deform capsule walls. Each therapeutic response is calculated to match the intensity of the detected anomaly, thereby providing a dosage-scaled, patient-specific intervention rather than a uniform release of active compounds.

The microencapsulated therapeutic agents consist of honey bee-derived substances—raw honey, propolis, and beeswax—encased in biodegradable polymer shells, with release characteristics governed by a combination of thermoresponsive and moisture-sensitive material layers. The control technique manages the spatial and temporal release of these agents through a zone-based activation matrix. This matrix divides the hydrogel layer into localized therapeutic zones, each with a unique sensor-actuator configuration and independently addressable control pathway. By comparing the sensor data within each zone to global and historical wound patterns, the technique selectively activates only those zones that require treatment, thereby conserving bioactive payload and preventing therapeutic oversaturation.

In addition to therapeutic control, the technique incorporates a logging module that timestamps each therapeutic event and records corresponding sensor metrics. This data is used to update internal healing models through an embedded self-learning loop. The system applies simple on-device gradient boosting or k-nearest-neighbor methods to refine future therapeutic actions based on response efficacy measured by wound parameter stabilization following each intervention. This allows the technique to “learn” from the patient-specific wound response, thereby evolving its intervention strategy over time.

The technique also includes a safeguard mechanism for therapeutic suppression. When all wound parameters fall within a predefined healing resolution band and remain stable for a minimum observation window (typically 12 to 24 hours), the technique suspends further therapeutic release and shifts to a passive monitoring mode. This is critical to prevent overtreatment and ensure natural tissue remodeling proceeds without unnecessary bioactive exposure. Should any parameters deviate from the safe band, the system automatically resumes therapeutic oversight.

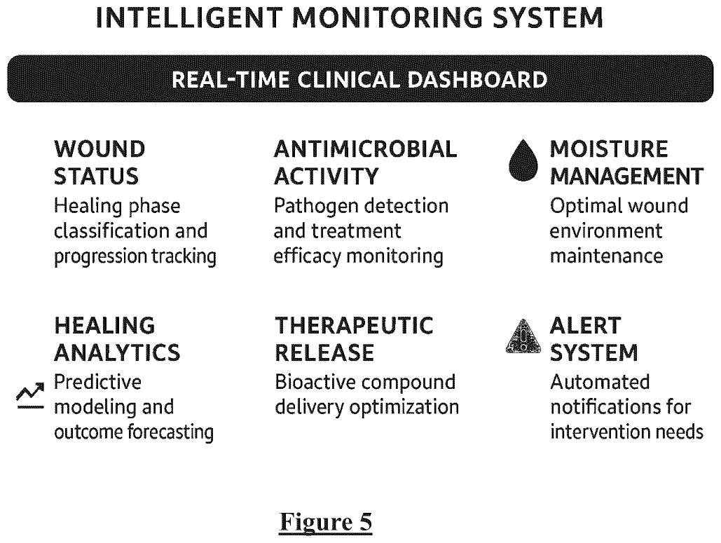

FIG. 5 illustrates the intelligent monitoring system integrated within the bioactive wound dressing specifically highlighting the functionalities of the real-time clinical dashboard. The dashboard serves as the central interface for continuously evaluating and adapting treatment parameters based on live wound site data. It includes a Wound Status module for healing phase classification and progression tracking, allowing precise assessment of the tissue recovery stage. The Antimicrobial Activity module enables detection of pathogenic agents and monitoring of treatment efficacy, while the Moisture Management module maintains optimal hydration levels essential for wound repair. The system also features a Therapeutic Release controller that dynamically regulates the delivery of bioactive compounds for enhanced therapeutic precision. In addition, the Healing Analytics module uses predictive modeling and outcome forecasting to refine treatment strategies based on historical and real-time data trends. To ensure timely medical response, an Alert System generates automated notifications whenever intervention thresholds are breached, ensuring proactive care delivery.

An optional wireless telemetry interface enables secure transmission of healing data logs, phase transition events, and therapeutic responses to an external receiver or clinician dashboard. This data can be used to remotely monitor healing trends, adjust therapeutic thresholds, or update the control technique firmware via over-the-air (OTA) mechanisms if new clinical models or improved therapeutic logic becomes available.

In terms of fabrication, each device is preloaded with a patient-specific treatment configuration profile based on wound type and clinical assessment, which seeds the initial parameter set for the technique. This configuration governs the initial release strategy, the sensitivity of sensor thresholds, and the zone activation matrix. As treatment proceeds, the firmware automatically fine-tunes these parameters based on feedback, creating a closed-loop, adaptive therapeutic system.

The technique underpinning this invention enables a precise, responsive, and personalized wound healing ecosystem, leveraging both deterministic rules and learning-based models. It continuously adapts therapeutic behavior to changing wound states while minimizing resource consumption and maximizing treatment efficacy. This computational framework transforms the wound dressing from a passive carrier of therapeutic substances into an intelligent, cyber-physical medical device capable of autonomous decision-making and therapeutic governance within the constraints of real-world clinical environments.

The wound dressing device comprises a machine-fabricated, multilayered scaffold structure that supports the sustained release of honey bee-derived therapeutic compounds across extended wound treatment periods. The top layer of the dressing is a breathable, antimicrobial membrane designed from biocompatible polymers with embedded propolis and silver nanoparticles. This layer provides initial protection against airborne contaminants and establishes a controlled interface for oxygen exchange and microbial control.

Beneath the top membrane lies a bioactive reservoir chamber fabricated from a biodegradable hydrogel matrix into which microcapsules are embedded. These microcapsules are composed of pH-sensitive or thermosensitive polymers enclosing formulations of raw honey, beeswax emulsions, and botanical adjuncts. The capsules are engineered using microfluidic or lithographic fabrication processes to ensure uniformity and reproducibility in release behavior. The microcapsules disintegrate in response to wound-specific triggers such as temperature elevation, enzymatic activity, or pH fluctuation, thereby releasing therapeutic agents in a phased manner tailored to healing stages.

A base layer made of sterile beeswax-coated cellulose functions as a thermal and antimicrobial shield, securing the dressing in place while maintaining an ideal moist wound environment. The beeswax component provides a natural barrier to external microbial invasion and assists in skin regeneration through its emollient and hydrophobic properties.

Integrated within the structure are microelectronic components forming a diagnostic layer. These may include biodegradable sensors, conductive hydrogels, or capacitive touch strips capable of detecting inflammation-related heat, wound pH, or exudate levels. Connected to these sensors is a passive circuitry that activates delivery modulation techniques, potentially via a Bluetooth low energy (BLE) interface for communication with external devices, such as clinical wound monitoring dashboards or mobile therapeutic management apps.

Therapeutic optimization is achieved via an embedded microcontroller unit (MCU) or logic gate array housed within an ultra-thin flexible circuit located at the posterior of the dressing. This unit receives data from embedded sensors and modulates the activation of embedded actuators, controlling microcapsule disintegration or responsive film expansion through electrothermal triggers. It also maintains an event log of antimicrobial release and healing progress.

The device's mechanical architecture is fabricated using additive manufacturing techniques such as 3D bioprinting or electrospinning, allowing for precision layering of bioactive and mechanical components. The entire dressing is sterilized post-manufacture using low-temperature ethylene oxide or gamma irradiation to preserve the bioactivity of honey-derived ingredients.

The system supports clinical integration through a modular docking system embedded at one edge of the device, which allows connection to handheld diagnostic readers or clinical monitoring tools. The dressing can be applied to wounds of various shapes and sizes due to its stretchable, conformable base layer, ensuring contact with irregular wound surfaces.

The invention relates to the field of bioactive medical devices, specifically to advanced wound care systems employing sustained-release delivery of therapeutic compounds derived from honey bees, including honey, propolis, and beeswax. It encompasses the domains of biomedical engineering, wound dressing technology, and intelligent therapeutic systems by integrating controlled microencapsulation, biosensing networks, embedded machine learning techniques, and adaptive release mechanisms. The invention further spans the technical areas of flexible electronics, biocompatible materials, and cyber-physical medical interfaces, providing a novel platform for real-time wound monitoring, antimicrobial regulation, and personalized therapeutic modulation within clinical, home care, and telemedicine environments.

The drawings and the forgoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, orders of processes described herein may be changed and are not limited to the manner described herein. Moreover, the actions of any flow diagram need not be implemented in the order shown; nor do all of the acts necessarily need to be performed. Also, those acts that are not dependent on other acts may be performed in parallel with the other acts. The scope of embodiments is by no means limited by these specific examples. Numerous variations, whether explicitly given in the specification or not, such as differences in structure, dimension, and use of material, are possible. The scope of embodiments is at least as broad as given by the following claims.

Benefits, other advantages, and solutions to problems have been described above with regard to specific embodiments. However, the benefits, advantages, solutions to problems, and any component(s) that may cause any benefit, advantage, or solution to occur or become more pronounced are not to be construed as a critical, required, or essential feature or component of any or all the claims.

1. A bioactive wound dressing device comprising:
a multilayered therapeutic architecture comprising at least a protective outer membrane, a bioactive hydrogel matrix, and a substrate contact layer;
wherein the bioactive hydrogel matrix comprises a plurality of microencapsulated honey bee-derived therapeutic agents selected from the group consisting of raw honey, propolis extract, and beeswax emulsions, each encapsulated within a biodegradable polymer shell; wherein the microencapsulated agents are configured for staged and sustained release based on wound-specific stimuli including at least one of pH, temperature, enzymatic activity, or moisture levels;
wherein the dressing further comprises an integrated microelectronic subsystem embedded between the outer membrane and the hydrogel matrix, said subsystem comprising biosensors configured to monitor wound healing indicators including pH and temperature, a logic control unit programmed to process sensor signals, and an adaptive release actuator configured to modulate the release rate of said microencapsulated therapeutic agents based on real-time wound conditions;
wherein the device is further configured to maintain a moist wound environment, enable continuous antimicrobial protection, and dynamically adjust therapeutic delivery parameters based on embedded feedback loops between sensor inputs and actuator outputs; wherein the raw honey encapsulated within the biodegradable polymer shell comprises a hydrogen peroxide concentration of at least 25 mMol/L and a glucose-to-fructose ratio between 0.85:1 and 1.15:1, wherein said composition enhances antimicrobial and osmotic activity upon release; and wherein the propolis extract comprises at least 30% total flavonoid content and exhibits a minimum inhibitory concentration (MIC) of less than 125 μg/mL against Staphylococcus aureus, Escherichia coli, and Pseudomonas aeruginosa in agar diffusion assays; and wherein the beeswax emulsions encapsulated within the hydrogel matrix possess a melting point in the range of 60-65° C. and function as a rheological modifier to increase the viscoelastic modulus of the hydrogel upon localized thermal stimulation; wherein the staged release of the microencapsulated agents follows a biphasic kinetic profile comprising an initial burst phase within 24-48 hours and a sustained zero-order phase extending up to 14 days, as governed by the degradation rate of the biodegradable polymer shell; and wherein the polymer shell is composed of polylactic-co-glycolic acid (PLGA) having a lactic-to-glycolic acid ratio between 75:25 and 50:50, and a molecular weight between 30-60 kDa; and wherein the biodegradable polymer shell enclosing the honey bee-derived therapeutic agents is selected from a group consisting of polylactic-co-glycolic acid (PLGA), polycaprolactone (PCL), or alginate derivatives, and wherein the shell composition and wall thickness are tuned during fabrication using microfluidic encapsulation to achieve time-variable release kinetics in accordance with therapeutic dosage profiles programmed into the logic control unit; and wherein the biosensor subsystem includes a printed, stretchable conductive hydrogel mesh embedded within the hydrogel matrix, said mesh configured to provide continuous measurement of wound-site bio-signals including electrical impedance, surface temperature fluctuations, and exudate ionic content, wherein said signals are transmitted to a microcontroller unit (MCU) for real-time analysis.

2. The device of claim 1, wherein the logic control unit comprises a flexible printed circuit incorporating a low-power MCU, non-volatile memory storage, and embedded therapeutic pattern recognition firmware, said firmware trained using supervised machine learning techniques to classify wound healing states and trigger activation or suppression of therapeutic compound release based on at least three wound state categories including inflammatory, proliferative, and remodeling phases; and wherein the adaptive release actuator comprises an electrothermal array or a magnetically actuated polymer network embedded within the hydrogel matrix, said actuator selectively activating microcapsule degradation or diffusion-based release in predefined spatial patterns aligned to wound healing gradients as calculated from biosensor-derived metrics.

3. The device of claim 1, wherein the protective outer membrane comprises a semi-permeable polyurethane layer impregnated with propolis nanoparticles and silver ions, configured to permit oxygen permeability while providing antimicrobial protection and mechanical shielding from environmental contaminants, and further comprising microperforations patterned via laser ablation to allow thermal and gaseous exchange without compromising sterility; and wherein the substrate contact layer comprises a beeswax-infused mesh composed of biodegradable cellulose or silk fibroin, configured to conform to irregular wound topographies, facilitate atraumatic removal upon dressing change, and promote epithelialization through controlled moisture retention and hydrophobic surface properties.

4. The device of claim 1, wherein the device further comprises a wireless communication module integrated within the logic control unit, said module selected from low-energy wireless protocols including Bluetooth Low Energy (BLE) or Near Field Communication (NFC), configured to transmit wound healing telemetry data, therapeutic history, and biosensor readings to external clinical monitoring systems for remote diagnostics and treatment optimization; and wherein the dressing device further comprises a therapeutic logging framework embedded within the logic unit firmware, said framework comprising timestamped records of therapeutic events including compound release quantities, sensor values, and healing state transitions, said data being stored locally in encrypted memory and optionally exported for forensic audit or compliance verification under clinical data governance protocols; and wherein the microencapsulation unit comprises a spatial distribution technique executed during dressing fabrication, said technique ensuring non-uniform dispersion density of microcapsules across the hydrogel matrix based on expected wound centerline healing delays, peripheral epithelialization rates, and historical wound treatment models stored in device memory.

5. The device of claim 1, wherein the biosensors embedded within the microelectronic subsystem further comprise microfabricated pH-sensitive field effect transistors (ISFETs) coated with polyaniline thin films, configured to detect wound acidity within the physiological range of 5.5 to 8.0 with a resolution of ±0.05 pH units; and wherein the adaptive release actuator includes a resistive electrothermal array patterned on a flexible polyimide substrate, wherein localized heating triggers degradation of thermoresponsive polymer coatings on select microcapsules.

6. The device of claim 1, wherein the logic control unit activates said electrothermal array based on sensor-derived thresholds for wound hydration, wherein release is initiated when moisture levels fall below 80% or exceed 95% relative humidity; wherein the logic control unit executes a trained neural network model comprising a long short-term memory (LSTM) architecture, configured to classify temporal wound healing patterns and generate control signals for the adaptive release actuator with prediction confidence above 90%; and wherein the multilayered therapeutic architecture is configured to degrade in situ over 14-21 days under physiological wound conditions, with over 90% mass loss occurring by day 18 in simulated wound fluid maintained at 35° C., thus synchronizing with the epithelialization timeline.

7. The device of claim 1, wherein the logic control unit further comprises a data fusion module configured to integrate simultaneous readings from the pH, temperature, and impedance sensors into a multidimensional wound condition vector, wherein said vector is analyzed using principal component analysis (PCA) prior to therapeutic decision-making; and wherein the wound condition vector is classified using a support vector machine (SVM) trained on at least 10,000 labeled wound states, said classifier outputting a phase label selected from an inflammatory phase, a proliferative phase, or a remodeling phase with greater than 92% classification accuracy.

8. The device of claim 1, wherein the adaptive release actuator is configured to support zone-based differential release, wherein wound zones exhibiting impaired healing receive higher localized dosages of therapeutic agents by selectively degrading adjacent microcapsule clusters; and wherein the microencapsulation unit comprises a dual-core shell structure, wherein an inner compartment encapsulates raw honey and an outer compartment encapsulates propolis extract, and wherein each compartment exhibits a distinct polymer degradation rate tuned by the fabrication flow rate ratio in a microfluidic device.

9. The device of claim 1, wherein propolis-containing compartment is composed of polycaprolactone (PCL) and the inner honey-containing compartment is composed of PLGA, wherein the difference in degradation profiles enables staged antimicrobial and anti-inflammatory delivery over 7-14 days; wherein the microelectronic subsystem further comprises an energy-harvesting unit configured to convert thermal gradients between the wound and the external environment into power via a flexible thermoelectric generator, said energy being used to intermittently power the biosensors and data logging module; and wherein the dressing further comprises a failsafe mechanism programmed into the logic control unit to enter a passive standby state and disable compound release when biosensor inputs fall outside physiological thresholds, indicating potential sensor malfunction or non-biological exposure.

10. The device of claim 1, wherein the therapeutic logging framework includes an embedded cryptographic hash engine that timestamps and encrypts each entry of therapeutic action, said log comprising compound ID, dose volume, release location, time of delivery, and biosensor context snapshot, wherein the biosensor subsystem is embedded within a conductive hydrogel mesh patterned in a hexagonal grid with inter-node distances between 2-4 mm, enabling spatial wound state mapping and vectorized signal propagation to the logic control unit; and wherein the microencapsulated therapeutic agents are non-uniformly distributed across the hydrogel matrix such that capsule density is highest near the predicted wound centroid, said distribution being derived from a radial healing model stored in the device firmware.

11. The device of claim 1, wherein the biodegradable polymer shell comprises a PLGA copolymer with a lactic-to-glycolic acid ratio of 65:35 and molecular weight of 50-70 kDa, wherein said polymer exhibits a hydrolytic degradation half-life of 4-6 days under wound-site pH conditions, enabling a quantized release profile programmable via capsule wall thickness control during microfluidic encapsulation; wherein the capsule wall thickness is varied between 200-500 nm across the matrix using flow-focusing microfluidic nozzles calibrated in real time via in-line Raman spectroscopy to achieve ±5% deviation from target diffusion rates stored in the logic control unit.

12. The device of claim 1, wherein the biosensor subsystem further comprises a 16-bit ADC module sampling at ≥100 Hz, and wherein impedance readings are passed through a Kalman filter and frequency-domain transformation before being used to calculate tissue hydration and inflammation metrics; and wherein the signal processing logic includes a real-time spectral entropy analysis of impedance time series, wherein high-entropy states correlate to necrotic tissue zones, triggering localized capsule degradation via spatial actuator activation, wherein the logic control unit executes a gated recurrent unit (GRU) neural network with five hidden layers, each comprising 32 units, trained on a dataset comprising at least 8,000 temporally annotated wound healing trajectories, said model configured to infer wound phase transitions with a confidence threshold ≥0.9 before initiating therapeutic release, and wherein the model weights are stored in encrypted flash memory and updated via secure OTA (over-the-air) firmware patches from a hospital-side diagnostic server.

13. The device of claim 1, wherein the adaptive release actuator is triggered through a feedback loop comprising (i) threshold detection logic, (ii) a proportional-integral-derivative (PID) controller tuned for individual wound response profiles, and (iii) an actuator activation queue optimized to avoid thermal saturation zones.

14. The device of claim 1, wherein the hydrogel matrix includes thermoresponsive channels patterned by soft lithography, said channels functioning as passive transport modulators whose permeability increases by 2-3× in response to wound temperature rising above 36.5° C.

15. The device of claim 1, wherein the adaptive release actuator is further configured to execute multi-dimensional dose prioritization based on real-time wound condition vectors derived from sensor fusion, said vectors comprising temporal pH gradients, sub-dermal thermal shifts, and impedance phase angle variances, wherein said actuator includes a tri-zonal microheating array fabricated using indium tin oxide (ITO) patterned on a polyimide substrate via photolithographic etching, and wherein each microheater is co-located with a thermosensitive microcapsule cluster encapsulated with a honey bee-derived agent of distinct function, such that raw honey is assigned to core inflammatory zones, propolis extract to oxidative stress margins, and beeswax emulsion to epithelialization fronts, the prioritization logic being determined by a predictive algorithm executed by the logic control unit, said algorithm comprising a hybrid convolutional recurrent neural network trained to infer wound zone function class from spatiotemporal sensor inputs and to sequentially trigger microheater actuation in order of therapeutic urgency with a temporal resolution below 30 seconds.

16. The device of claim 1, wherein the logic control unit is further programmed to implement a closed-loop feedback optimization protocol comprising: (i) real-time wound state classification into five microphases using an LSTM-based inference engine; (ii) therapeutic capsule depletion tracking using a dynamic lookup table indexed by capsule type, location, prior release timestamp, and actuator trigger count; (iii) long-range depletion forecasting via a forward-modeling Kalman predictor trained on at least 2,000 wound treatment datasets; and (iv) actuation efficiency scoring derived from feedback success correlation between predicted and actual healing progression, wherein said optimization loop is executed once every 15 minutes, logged to encrypted firmware memory in 256-bit SHA-encrypted blocks, and used to adjust control parameters including activation delay (td), actuator pulse width (pw), and maximum spatial redundancy (Rmax) such that the therapeutic delivery strategy converges on a clinically optimal release envelope while preserving remaining microcapsule density above 15% until wound closure is predicted, with all loop parameters tunable via authenticated remote clinician interface through a Bluetooth Low Energy (BLE) telemetry module embedded in the logic control unit.
